(19) United States
(12) Patent Application Publication
(10) Pub. No.: US 2025/0206687 A1
(43) Pub. Date: Jun. 26, 2025

(54) METHODS FOR CONVERTING INTERMEDIATES IN PROCESSES FOR PRODUCING trans-1,2-DIFLUOROETHYLENE (HFO-1132E)

(71) Applicant: Honeywell International Inc., Charlotte, NC (US)

(72) Inventors: Akbar Mahdavi-Shakib, Buffalo, NY (US); Haiyou Wang, Amherst, NY (US)

(21) Appl. No.: 18/988,359

(22) Filed: Dec. 19, 2024

Related U.S. Application Data
(60) Provisional application No. 63/613,982, filed on Dec. 22, 2023.
(60) Provisional application No. 63/718,445, filed on Nov. 8, 2024.
(60) Provisional application No. 63/724,856, filed on Nov. 25, 2024.

(51) Int. Cl. C07C 17/20; B01D 3/14; B01J 23/44; C07C 17/25

(52) U.S. Cl. CPC C07C 17/202; B01D 3/143; B01J 23/44; C07C 17/25

(57) ABSTRACT

In a first step reaction for producing 1,1,2-trifluoroethane (HFC-143) from 1,1,2-trichloro-1,2,2-trifluoroethane (CFC-113) as part of an overall method for production of trans-1,2-difluoroethylene (HFO-1132E), several intermediates and/or byproducts are formed, some of which may be considered desired and others undesired. The overall reaction methods and/or specific reactions conditions for producing 1,1,2-trifluoroethane (HFC-143) from 1,1,2-trichloro-1,2,2-trifluoroethane (CFC-113) may be selectively tailored to usefully convert desired intermediates to the desired product 1,1,2-trifluoroethane (HFC-143) and/or minimize the formation of undesired byproducts.

### CROSS-REFERENCE TO RELATED APPLICATIONS

This application claims the benefit under 35 U.S.C. § 119(e) of U.S. Provisional Application No. 63/613,982 entitled “METHODS FOR CONVERTING INTERMEDIATES IN PROCESSES FOR PRODUCING trans-1,2-DIFLUOROETHYLENE (HFO-1132E)”, filed on Dec. 22, 2023, Provisional Application No. 63/718,445 entitled “METHODS FOR CONVERTING INTERMEDIATES IN PROCESSES FOR PRODUCING trans-1,2-DIFLUOROETHYLENE (HFO-1132E)”, filed on Nov. 8, 2024, and Provisional Application No. 63/724,856 entitled “METHODS FOR CONVERTING INTERMEDIATES IN PROCESSES FOR PRODUCING trans-1,2-DIFLUOROETHYLENE (HFO-1132E)”, filed on Nov. 25, 2024, the entire disclosures of all three are incorporated by reference in their entireties.

### FIELD

The present disclosure relates generally to a method for producing trans-1,2-difluoroethylene (HFO-1132E) from 1,1,2-trichloro-1,2,2-trifluoroethane (CFC-113), and more specifically to methods for managing the formation of and/or converting intermediates that may be formed in the HFO-1132E process.

### BACKGROUND

1,2-difluoroethylene (HFO-1132) has recently found increased utility for a variety of uses. HFO-1132 may exist as a mixture of two geometric isomers, the E- or trans isomer and the Z- or cis isomer, which may be used separately or together in various proportions. Potential end use applications of HFO-1132 include refrigerants, either used alone or in blends with other components, solvents for organic materials, and as a chemical intermediate in the synthesis of other halogenated hydrocarbon solvents.

Certain intermediates and/or byproducts are produced in the process for manufacturing HFO-1132. It would be desirable to convert any useful intermediates into desired products and/or minimize formation of any undesired byproducts in the process to produce the desired product HFO-1132.

### SUMMARY

HFO-1132 and, in particular, HFO-1132E, is produced from 1,1,2-trichloro-1,2,2-trifluoroethane (CFC-113). In a first step, 1,1,2-trifluoroethane (HFC-143) is produced by hydrogenating CFC-113 by reaction with hydrogen in the presence of a catalyst to produce HFC-143. The HFC-143 is then dehydrofluorinated in the presence of a catalyst to produce trans-1,2-difluoroethylene (HFO-1132E) and/or cis-1,2-difluoroethylene (HFO-1132Z). The HFO-1132Z may then optionally be isomerized to produce HFO-1132E.

It has been found that, in the first step for producing HFC-143 from CFC-113, several intermediates and/or byproducts are formed, some of which are considered desired intermediates and others undesired byproducts. The present disclosure is based on the discovery that the overall reaction methods and/or specific reaction conditions of the first step for producing HFC-143 from CFC-113 may be selectively tailored to advantageously convert desired intermediates to the desired product HFC-143 and/or minimize the formation of undesired byproducts.

In one form thereof, the present disclosure provides a method for producing HFC-143, comprising: reacting at least one of CFC-113, 1-chloro-1,1,2-trifluoroethane (HCFC-133b), 1-chloro-1,2,2-trifluoroethane (HCFC-133) and 1,2-dichloro-1,1,2-trifluoroethane (HCFC-123a) in the presence of a catalyst at a temperature greater than 150° C. to produce HFC-143.

In another form thereof, the present disclosure provides a method for producing HFC-143, comprising: hydrogenating CFC-113 by reaction with hydrogen to produce a first product composition comprising HFC-143 and at least one of HCFC-133b, HCFC-133 and HCFC-123a; and hydrogenating at least one of HCFC-133b, HCFC-133 and HCFC-123a from the first product composition in the presence of a catalyst at a temperature greater than 150° C. to produce a second product composition comprising HFC-143.

### BRIEF DESCRIPTION OF THE DRAWINGS

FIG. 1 is a schematic diagram of a single reactor layout for the conversion of 1,1,2-trichloro-1,2,2-trifluoroethane (CFC-113) to 1,1,2-trifluoroethane (HFC-143).

FIG. 2 is a schematic diagram of a two-reactor layout for the conversion of CFC-113 to HFC-143.

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” or “any range using any two of the foregoing values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value. For example, a range of as low as 1, 2, or 3, or as high as 8, 9, or 10 followed by this phrase encompasses ranges including 1 to 10, or 2 to 8, or 3 to 9.

In the Examples below, the designation “R” may be used in connection with the various fluorine-containing molecules described herein, for example, “R-143” refers to 1,1,2-trifluoroethane (HFC-143).

As used herein, the phrase “desired product” is 1,1,2-trifluoroethane (HFC-143).

As used herein, the phrase “desired intermediates” include one or more of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a).

As used herein, the phrase “undesired byproducts” include one or more of 1,1,1-trifluoroethane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)).

As used herein, the phrase “based on total moles of organic components of the composition” refers only to carbon-containing components and does not include or encompass non-carbon-containing components such as hydrogen (H₂) or hydrogen chloride (HCl).

As used herein, conversion of a reactant molecule (molecule X) during a reaction is calculated using the following equation:

$$\% selectivity to molecule X = mol . \% of molecule X in the organic components of a \\ product mixture / ( 100 - mol . \% of reactant molecule \\ in the organic components of a product mixture ) \times 100.$$

As used herein, selectivity to a molecule formed during a reaction (molecule X) is calculated using the following equation:

$$\% conversion of molecule X = ( 100 - molecule X mol . \% in the organic components of a product mixture )$$

As used herein, amount of increase in a molecule formed after a second reacting step in a second product mixture (molecule X) compared to an amount of such molecule in a first product mixture formed in a first reacting step is calculated using the following equation:

$$\% increase in molecule X = ( ( mol . \% of molecule X in the organic components \\ of a second product mixture - mol . \% of molecule \\ X in the organic components of a first product mixture ) / \\ ( mol . \% of molecule X in the organic components \\ of the first product mixture ) / ( mol . \% of molecule \\ X in the organic components of the first product mixture ) ) \times 100.$$

### II. Overview

The present disclosure provides a method for producing E-1,2-difluoroethylene (HFO-1132E) from 1,1,2-trichloro-1,2,2-trifluoroethane (CFC-113) according to a three-step process shown below (“Process 1”), which includes the following three steps: (i) hydrogenating CFC-113 to produce 1,1,2-trifluoroethane (HFC-143), (ii) dehydrofluorinating HFC-143 to produce a mixture of trans-1,2-difluoroethylene (HFO-1132E) and cis-1,2-difluoroethylene (HFO-1132Z), and (iii) optionally isomerizing HFO-1132Z to HFO-1132E.

Schematic equations for the three steps of Process 1 are represented below:

### Process 1

CFCl₂—CF₂Cl(CFC-113)+H₂→CFH₂—CF₂H(HFC-143)+HCl (i)

CFH₂—CF₂H+trans-CFH=CHF(HFO-1132E)+cis-CFH=CFH(HFO-1132Z)+HF (ii)

cis-CFH=CFH(HFO-1132Z)+trans-CFH=CHF(HFO-1132E) (iii)

Step (i) may proceed through an intermediate of 1,1,2-trifluoroethene (HFO-1123), wherein CFC-113 is first hydrogenated to produce HFO-1123 as an intermediate, which is itself then hydrogenated to produce HFC-143.

Further, as discussed herein, it has been found that, in Step (i), several intermediates and/or byproducts are formed, some of which may be considered desired intermediates and others undesired byproducts, and it has been found that the overall reaction methods and/or specific reaction conditions of Step (i) may be selectively tailored to advantageously convert desired intermediates to the desired product HFC-143 and/or minimize the formation of undesired byproducts.

Further details regarding each of Steps (i), (ii), (iii) are set forth below.

### III. Step (i)

### General Process

The hydrogenation reaction of Step (i) may be carried out in the gas or vapor phase in a suitable reactor, for example a tubular reactor made from a material which is resistant to temperature and/or corrosion such as nickel and its alloys, including Hastelloy (for example, Hastelloy C276), Inconel (for example, Inconel 600), Incoloy, and Monel, and the vessels may be lined with fluoropolymers.

The reactor used for the hydrogenation reaction of Step (i) may be first cleaned and flushed with an inert gas such as nitrogen, followed by packing with a catalyst such as those described below. The catalyst may be pretreated within the reactor such as by drying in the manner described further below, followed by metering the reactants into the reactor to initiate the reaction.

The process flow for the hydrogenation reaction of Step (i) may be in the down or up direction through a bed of the catalyst. Products may be flowed through one or more scrubbers to remove undesired byproducts from the reaction, such as hydrogen fluoride (HF) and/or hydrogen chloride (HCl), and the reaction products may be collected by capture in a cooled cylinder, for example.

As discussed in further detail below, the catalyst and process conditions play an important role in the hydrogenation reaction of Step (i).

### Catalysts

In the hydrogenation reaction of Step (i), the catalyst may comprise a metal such as palladium, platinum, rhodium, ruthenium, iron, cobalt or nickel. Specifically, the catalyst active to catalyze the reaction may preferably be palladium metal (Pd), platinum metal (Pt), or a combination of palladium metal and platinum metal.

### Catalyst Support

In the hydrogenation reaction of Step (i), the catalyst may be supported on a suitable support, such as carbon or alumina (aluminum oxide—Al₂O₃). The carbon may be activated carbon. The alumina may be alpha(α) alumina, theta(θ) alumina, delta(δ) alumina, or gamma(γ) alumina. The supported catalyst may be produced by impregnation of any of the suitable supports with a solution of a compound of the desired metal constituent. The support may also be in the form of pellets. After the impregnation step, the solvent may be removed using heat or under vacuum resulting in a solid mass which can be further dried, calcined, and reduced to form active metal catalyst.

In the hydrogenation reaction of Step (i), the catalyst may be palladium on a carbon support, may be platinum on a carbon support, may be rhodium on a carbon support, and/or may be palladium, platinum, or rhodium on an alumina support. The catalyst may be palladium on a carbon support. The catalyst may be palladium on an alpha alumina support.

In the hydrogenation reaction of Step (i), the metal catalysts supported on various catalyst supports used are listed in Table 1 below.

TABLE 1
Supported Metal Catalysts - Step (i) Hydrogenation Reaction

| Catalyst | Support |
|---|---|
| Pd | Activated Carbon |
| Pd | alpha(α)-Al₂O₃ |
| Pd | theta(θ)-Al₂O₃ |
| Pd | delta(δ)-Al₂O₃ |
| Pd | gamma(γ)-Al₂O₃ |
| Pt | Activated Carbon |
| Pt | alpha(α)-Al₂O₃ |
| Pt | theta(θ)-Al₂O₃ |
| Pt | delta(δ)-Al₂O₃ |
| Pt | gamma(γ)-Al₂O₃ |
| Rh | Activated Carbon |
| Rh | alpha(α)-Al₂O₃ |
| Rh | theta(θ)-Al₂O₃ |
| Rh | delta(δ)-Al₂O₃ |
| Rh | gamma(γ)-Al₂O₃ |

### Catalyst Loading

For each catalyst/support combination (each row) in Table 1 used in the hydrogenation reaction of Step (i), the amount of metal loading on the support is from as little as about 0.01 wt. %, about 0.05 wt. %, about 0.1 wt. %, about 0.2 wt. %, about 0.3 wt. %, about 0.4 wt. %, about 0.5 wt. %, about 1 wt. %, or as great as about 2 wt. %, about 3 wt. %, about 5 wt. %, about 10 wt. %, about 20 wt. %, about 30 wt. %, about 40 wt. %, about 50 wt. %, based on a total weight of the catalyst and support, or within any range encompassed by two of the foregoing values as endpoints, for example, from about 0.01 wt. % to about 20 wt. %, from about 0.01 wt. % to about 10 wt. %, from about 0.1 wt. % to about 10 wt. %, 0.2 wt. % to about 10 wt. %, from about 0.5 wt. % to about 5 wt. %, from about 1 wt. % to about 5 wt. %, from about 1 wt. % to about 4 wt. %, or about 2 wt. % to about 4 wt. %, based on a total weight of the catalyst and support. For supported noble metal catalysts such as Pd or Pt or Rh, the metal loading may be from about 0.01 wt. % to about 5 wt. % based on a total weight of the catalyst and support. Specific examples of additional suitable ranges are set forth below in Table 2.

TABLE 2
Loading for Catalysts (Pd, Pt, Rh) on Supports (Activated Carbon,
alpha(α)-Al₂O₃, theta(θ)-Al₂O₃, delta(δ)-Al₂O₃, or
gamma(γ)-Al₂O₃) - Each Catalyst/Support Combination (Each
Row) in Table 1 - Step (i) Hydrogenation Reaction

| From (wt. %) | To (wt. %) |
|---|---|
| 0.01 | 20 |
| 0.01 | 10 |
| 0.01 | 5 |
| 0.01 | 4 |
| 0.5 | 20 |
| 0.5 | 10 |
| 0.5 | 5 |
| 0.5 | 4 |
| 1 | 20 |
| 1 | 10 |
| 1 | 5 |
| 1 | 4 |
| 2 | 20 |
| 2 | 10 |
| 2 | 5 |
| 2 | 4 |
| 0.1 | 0.2 |
| 0.2 | 0.3 |
| 0.3 | 0.4 |
| 0.4 | 0.5 |
| 0.5 | 1 |
| 1 | 2 |

### BET Surface Area

The BET (Brunauer, Emmet, and Teller) analysis is the standard method for determining surface areas from nitrogen adsorption isotherms. The BET surface areas of catalysts may be measured using TriStar II Micromeritics instrument. Catalyst samples are degassed using FlowPrep 060 instrument before BET analysis.

For each catalyst/support combination (each row) in Table 1 used in the hydrogenation reaction of Step (i), the BET surface area may be as low as about 0.5 m²/g, about 1 m²/g, about 3 m²/g, about 5 m²/g, about 10 m²/g, about 15 m²/g, about 20 m²/g², about 30 m²/g, about 40 m²/g, about 50 m²/g, about 100 m²/g, about 200 m²/g, or as high as about 250 m²/g, about 300 m²/g, about 400 m²/g, about 500 m²/g, about 600 m²/g, about 700 m²/g m², about 800 m²/g, about 900 m²/g, about 1000 m²/g, about 2000 m²/g, about 3000 m²/g, or within any range encompassed by any of the foregoing values as endpoints, from example, from about 0.5 m²/g to about 3000 m²/g, from about 1 m²/g to about 2000 m²/g, from about 1000 m²/g to about 2000 m²/g, from about 0.5 m²/g to about 500 m²/g, from about 0.5 m²/g to about 300 m²/g, from about 0.5 m²/g to about 5 m²/g, or from about 200 m²/g to about 300 m²/g.

For carbon supported metal catalysts (Pd, Pt, Rh) used in the hydrogenation reaction of Step (i), the BET surface area may be from about 100 m²/g to about 3000 m²/g, preferably from about 200 m²/g to about 2000 m²/g, more preferably from about 500 m²/g to about 1500 m²/g, and most preferably from about 1000 m²/g to about 1500 m²/g. Specific examples of additional suitable ranges are set forth below in Table 3a.

For alumina (alpha(α)-Al₂O₃, theta(θ)-Al₂O₃, delta(δ)-Al₂O₃, or gamma(γ)-Al₂O₃) supported metal catalysts (Pd, Pt, Rh) used in the hydrogenation reaction of Step (i), the BET surface area may be from about 0.5 m²/g to about 500 m²/g, preferably from about 1 m²/g to about 200 m²/g, more preferably from about 1 m²/g to about 100 m²/g, and most preferably from about 1 m²/g to about 20 m²/g. Specific examples of additional suitable ranges are set forth below in Table 3b.

TABLE 3a
BET Surface Area of Catalysts (Pd, Pt, Rh) on Supports
(Activated Carbon) - Step (i) Hydrogenation Reaction

| From (m²/g) | To (m²/g) |
|---|---|
| 100 | 3000 |
| 100 | 2000 |
| 100 | 1500 |
| 200 | 3000 |
| 200 | 2000 |
| 200 | 1500 |
| 500 | 3000 |
| 500 | 2000 |
| 500 | 1500 |
| 1000 | 3000 |
| 1000 | 2000 |
| 1000 | 1500 |
| 100 | 200 |
| 200 | 500 |
| 500 | 1000 |
| 1000 | 1500 |
| 1500 | 2000 |
| 2000 | 3000 |

TABLE 3b
BET Surface Area of Catalysts (Pd, Pt, Rh) on Supports (alpha(α)-Al₂O₃,
theta(θ)-Al₂O₃, delta(δ)-Al₂O₃, or gamma(γ)-Al₂O₃) -
Step (i) Hydrogenation Reaction

| From (m²/g) | To (m²/g) |
|---|---|
| 0.5 | 500 |
| 0.5 | 200 |
| 0.5 | 100 |
| 0.5 | 20 |
| 1 | 500 |
| 1 | 200 |
| 1 | 100 |
| 1 | 20 |
| 0.5 | 1 |
| 1 | 50 |
| 50 | 100 |
| 100 | 200 |
| 200 | 500 |

### Catalyst Pretreatment

For each catalyst/support combination (each row) in Table 1 used in the hydrogenation reaction of Step (i), the catalyst may be pretreated by a variety of methods to improve its performance and effectiveness in the reaction. For example, the catalyst may be dried at elevated temperatures, as low as about 200° C., about 250° C., about 300° C., about 350° C., about 360° C., about 370° C., or as high as about 380° C., about 390° C., about 400° C., about 450° C., about 500° C., about 600° C., about 700° C., or within any range encompassed by two of the foregoing values as endpoints, such as from about 200° C. to about 400° C., from about 200° C. to about 350° C., from about 250° C. to about 350° C., from about 250° C. to about 300° C., or from about 260° C. to about 300° C. Specific examples of additional suitable ranges are set forth below in Table 4.

TABLE 4
Pre-treatment Drying Temperature for Catalysts (Pd, Pt, Rh) on Supports
(Activated Carbon, alpha(α)-Al₂O₃, theta(θ)-Al₂O₃, delta(δ)-Al₂O₃,
or gamma(γ)-Al₂O₃) - Each Catalyst/Support Combination (Each Row) in
Table 1 - Step (i) Hydrogenation Reaction

| From (° C.) | To (° C.) |
|---|---|
| 200 | 400 |
| 200 | 350 |
| 200 | 300 |
| 220 | 400 |
| 220 | 350 |
| 220 | 300 |
| 250 | 400 |
| 250 | 350 |
| 250 | 300 |
| 260 | 400 |
| 260 | 350 |
| 260 | 300 |

For each catalyst/support combination (each row) in Table 1 used in the hydrogenation reaction of Step (i), as part of the catalyst pretreatment, the catalyst (Pd, Pt, Rh) may be exposed to an inert gas such as N₂. The pretreatment process may take as low as about 1 hour, about 2 hours, about 3 hours, about 4 hours, or as high about 5 hours, about 6 hours, about 10 hours, about 20 hours, or within any range encompassed by two of the foregoing values as endpoints such as about 2 hours to about 4 hours, for example from about 1 hour to about 20 hours, from about 2 hours to about 10 hours, from about 3 hours to about 6 hours, or from about 4 hours to about 5 hours.

### Reaction Condition—Temperature

For reactions using each catalyst/support combination (each row) in Table 1, the reaction temperature of the hydrogenation reaction of Step (i) may be as low as about 100° C., about 125° C., about 150° C., about 200° C., about 250° C. or as high as about 300° C., about 350° C., about 400° C., or within any range encompassed by two of the foregoing values as endpoints, such as from about 100° C. to about 400° C., or from about 125° C. to about 350° C., from about 150° C. to about 300° C., or from about 200° C. to about 250° C., for example. The temperature may be preferably from about 100° C. to about 350° C., and more preferably from about 200° C. to about 300° C. Specific examples of additional suitable ranges are set forth below in Table 5.

TABLE 5
Reaction Temperature When Using Catalysts (Pd, Pt, Rh) on Supports
(Activated Carbon, alpha(α)-Al₂O₃, theta(θ)-Al₂O₃, delta(δ)-Al₂O₃,
or gamma(γ)-Al₂O₃) - Each Catalyst/Support Combination (Each
Row) in Table 1 - Step (i) Hydrogenation Reaction

| From (° C.) | To (° C.) |
|---|---|
| 100 | 400 |
| 100 | 350 |
| 100 | 300 |
| 100 | 250 |
| 100 | 200 |
| 100 | 150 |
| 100 | 125 |
| 125 | 400 |
| 125 | 350 |
| 125 | 300 |
| 125 | 250 |
| 125 | 200 |
| 125 | 150 |
| 150 | 400 |
| 150 | 350 |
| 150 | 300 |
| 150 | 250 |
| 150 | 200 |
| 200 | 400 |
| 200 | 350 |
| 200 | 300 |
| 200 | 250 |
| 250 | 400 |
| 250 | 350 |
| 250 | 300 |
| 300 | 400 |
| 300 | 350 |
| 350 | 400 |

As demonstrated by the Examples herein, the selectivity towards the desired product 1,1,2-trifluoroethane (HFC-143) of the hydrogenation reaction of Step (i) may increase with temperature. However, the overall selectivity to 1,1,2-trifluoroethane (HFC-143) and its associated recyclable intermediates such as 1-chloro-1,1,2-trifluoroethane (HCFC-133b), 1-chloro-1,2,2-trifluoroethane (HCFC-133), 1,2-dichloro-1,1,2-trifluoroethane (HCFC-123a), and trifluoroethylene (HFO-1123) may decrease with high temperature because of increased formation of undesired byproducts such as ethane (HC-170), chloroethane (HCC-160), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), 1,1-difluoroethane (HFC-152a), 1,1,1-trifluoroethane (HFC-143a), etc.

### Reaction Condition—Contact Time

For reactions using each catalyst/support combination (each row) in table 1, the contact time of the reactants with the catalyst (Pd, Pt, Rh) in the hydrogenation reaction of Step (i) may be as little as about 0.1 second, about 1 second, about 2 seconds, about 5 seconds, about 10 seconds, about 15 seconds, about 20 seconds, or as long as about 25 seconds, about 30 seconds, about 40 seconds, about 50 seconds, about 60 seconds, about 80 seconds, about 120 seconds, or within any range encompassed by two of the foregoing values as endpoints, such as from about 0.1 seconds to about 120 seconds, from about 1 second to about 60 seconds, from about 5 seconds to about 50 seconds, from about 10 seconds to about 40 seconds, from about 15 seconds to about 30 seconds, or about 20 seconds to about 25 seconds. For example, the contact time may be from about 1 second to about 60 seconds. Specific examples of additional suitable ranges are set forth below in Table 6.

TABLE 6
Contact Time of Reactants with Catalysts (Pd, Pt, Rh) on Supports
(Activated Carbon, alpha(α)-Al₂O₃, theta(θ)-Al₂O₃, delta(δ)-Al₂O₃,
or gamma(γ)-Al₂O₃) - Each Catalyst/Support Combination (Each
Row) in Table 1 - Step (i) Hydrogenation Reaction

| From (seconds) | To (seconds) |
|---|---|
| 1 | 120 |
| 1 | 100 |
| 1 | 80 |
| 1 | 60 |
| 1 | 40 |
| 1 | 20 |
| 5 | 120 |
| 5 | 100 |
| 5 | 80 |
| 5 | 60 |
| 5 | 40 |
| 5 | 20 |
| 10 | 120 |
| 10 | 100 |
| 10 | 80 |
| 10 | 60 |
| 10 | 40 |
| 10 | 20 |
| 0.1 | 1 |
| 1 | 2 |
| 2 | 5 |
| 5 | 10 |
| 20 | 120 |
| 20 | 100 |
| 20 | 80 |
| 20 | 60 |
| 20 | 40 |
| 40 | 50 |
| 50 | 60 |
| 60 | 70 |
| 70 | 80 |
| 80 | 100 |
| 100 | 120 |

### Reaction Condition—Pressure

For reactions using each catalyst/support combination (each row) in Table 1, the pressure inside which reactor the hydrogenation reaction of Step (i) takes place may be as little as about 1 psig, about 3 psig, about 5 psig, about 10 psig, about 15 psig, about 20 psig, about 30 psig, about 35 psig or about 40 psig, or as great as about 90 psig, about 100 psig, about 120 psig, about 150 psig, about 200 psig or about 250 psig, about 300 psig, or within any range encompassed by two of the foregoing values as endpoints, such as from about 1 psig to about 300 psig, from about 3 psig to about 250 psig, from about 5 psig to about 200 psig, from about 10 psig to about 150 psig, from about 15 psig to about 120 psig, from about 20 psig to about 100 psig, from about 30 psig to about 90 psig, or from about 35 psig to about 40 psig. For example, the pressure may be from about 10 psig to about 200 psig. Specific examples of additional suitable ranges are set forth below in Table 7.

TABLE 7
Reaction Pressure using Catalysts (Pd, Pt, Rh) on Supports
(Activated Carbon, alpha(α)-Al₂O₃, theta(θ)-Al₂O₃, delta(δ)-Al₂O₃,
or gamma(γ)-Al₂O₃) - Each Catalyst/Support Combination (Each
Row) in Table 1 - Step (i) Hydrogenation Reaction

| From (psig) | To (psig) |
|---|---|
| 1 | 300 |
| 1 | 250 |
| 1 | 200 |
| 1 | 150 |
| 1 | 120 |
| 1 | 100 |
| 1 | 90 |
| 1 | 40 |
| 15 | 300 |
| 15 | 250 |
| 15 | 200 |
| 15 | 150 |
| 15 | 120 |
| 15 | 100 |
| 35 | 300 |
| 35 | 250 |
| 35 | 200 |
| 35 | 150 |
| 35 | 120 |
| 35 | 100 |
| 35 | 90 |
| 35 | 40 |
| 100 | 300 |
| 100 | 250 |
| 100 | 200 |
| 100 | 150 |
| 100 | 120 |
| 200 | 300 |
| 200 | 250 |
| 250 | 300 |

### Reaction Conditions—Hydrogen Mole Ratio

For reactions using each catalyst/support combination (each row) in Table 1 used in the hydrogenation reaction of Step (i), the mole ratio of hydrogen to CFC-113 may be as little about 2:1, about 3:1, about 4:1, about 5:1, about 5.5:1 or as great as about 6:1, about 6.5:1, about 7.5:1 or about 8:1, about 12:1, about 15:1, or about 20:1, for example, or within any range encompassed by two of the foregoing values as endpoints. The mole ratio of hydrogen to CFC-113 may be preferably from about 3:1 about to 15:1, and more preferably from about 4:1 to about 10:1.

### Products—Selectivity to HFC-143

As demonstrated by the Examples herein, for reactions using each catalyst/support combination (each row) in Table 1, the hydrogenation Step (i) may achieve a selectivity to the desired product 1,1,2-trifluoroethane (HFC-143) product of at least about 20%, at least about 30%, at least about 40%, at least about 50%, at least about 60%, at least about 70%, at least about 80%, at least about 90%, and for each of the foregoing, less than or equal to 100%, or within any range encompassed by two of the foregoing values as endpoints, such as from about 20% to about 90%, from about 30% to about 80%, from about 40% to about 80%, or about 50% to about 80%, based on total moles of the organic components of the composition. Specific examples of additional suitable ranges are set forth below in Table 8.

TABLE 8
Selectivity to Desired Product HFC-143 using Catalyst (Pd, Pt, Rh) on
Supports (Activated Carbon, alpha(α)-Al₂O₃, theta(θ)-Al₂O₃,
delta(δ)-Al₂O₃, or gamma(γ)-Al₂O₃) - Each Catalyst/Support
Combination (Each Row) in Table 1 - Step (i) Hydrogenation Reaction

| From (%) | To (%) |
|---|---|
| 20 | 90 |
| 20 | 80 |
| 20 | 70 |
| 20 | 60 |
| 30 | 80 |
| 30 | 70 |
| 30 | 60 |
| 40 | 80 |
| 40 | 70 |
| 40 | 60 |
| 40 | 50 |
| 50 | 60 |
| 60 | 70 |
| 70 | 80 |
| 80 | 90 |

### Methods for Conversion of Intermediates

As discussed below, during the hydrogenation reaction of Step (i), several intermediates and/or byproducts may be formed, some of which may be considered desired intermediates and others undesired byproducts, wherein the overall reaction methods and/or specific reaction conditions of Step (i) may be selectively tailored to usefully convert desired intermediates to the product 1,1,2-trifluoroethane (HFC-143) and/or minimize the formation of undesired byproducts.

In particular, the hydrogenation reaction of Step (i) for producing 1,1,2-trifluoroethane (HFC-143) from 1,1,2-trichloro-1,2,2-trifluoroethane (CFC-113), using each catalyst/support combination (each row) in Table 1, may produce several desired intermediates such as 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a), as well as several undesired byproducts such as 1,1,1-trifluorothane (HFC-143a), ethane (HC-170), chloroethane (HCC-160), 1,1-difluoroethane (HFC-152a), and/or HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)).

It has been discovered that the intermediates 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a) are formed in the hydrogenation reaction of Step (i) using each catalyst/support combination (each row) in Table 1, and that each of 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a) are less reactive than 1,1,2-trichloro-1,2,2-trifluoroethane (CFC-113) for producing 1,1,2-trifluoroethane (HFC-143).

However, notwithstanding the foregoing, it has also been found that each of 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a) may themselves be converted to 1,1,2-trifluoroethane (HFC-143) via the methods and reaction conditions herein and, in view of this finding, 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a) may be considered “desired intermediates” because each of the foregoing may be converted to the desired product 1,1,2-trifluoroethane (HFC-143) in order to increase the overall efficiency of the Step (i) process using each catalyst/support combination (each row) in Table 1.

For example, the foregoing desired intermediates, when formed in the hydrogenation reaction of Step (i) using each catalyst/support combination (each row) in Table 1, are potentially recyclable as reactants to the hydrogenation reaction and can be further reacted to form 1,1,2-trifluoroethane (HFC-143). However, recycling these intermediates is potentially difficult because such intermediates are less reactive than 1,1,2-trichloro-1,2,2-trifluoroethane (CFC-113) and therefore may potentially accumulate or build up in the reactor and/or elsewhere in the overall process architecture, thereby hindering selectivity, yield, and purity of the desired product 1,1,2-trifluoroethane (HFC-143). The reaction conditions and layouts disclosed herein are designed to promote recycling and further reaction of the foregoing intermediates to result in formation of the desired 1,1,2-trifluoroethane (HFC-143).

The present disclosure provides a single-reactor method and a two-reactor method to promote the conversion of the intermediates to the desired product 1,1,2-trifluoroethane (HFC-143).

i. Single-Reactor Method

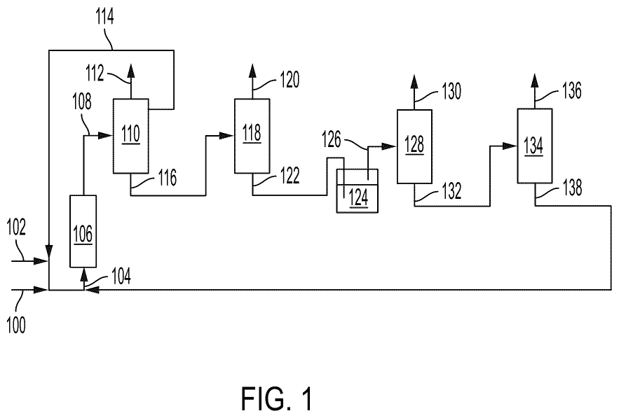

In the single-reactor method, the overall reaction method will be described in more details below in connection with FIG. 1, though in connection with certain aspects described below, the reaction conditions may be selectively tailored to maximize the production of desired products and intermediates and to minimize the production of undesired byproducts, wherein the reactor in a single-reactor method may be used with the reaction carried out at a higher temperature and using a more reactive catalyst as compared to the conditions described above.

As demonstrated by the Examples herein, increasing the reaction temperature and/or using a more reactive catalyst promotes conversion of the less reactive, yet desired intermediates including 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) to the desired product 1,1,2-trifluoroethane (HFC-143).

Referring to FIG. 1, stream 100 comprising CFC-113 and stream 102 comprising hydrogen (H₂) enter reactor 106. Inside reactor 106, the CFC-113 undergoes a Step (i) hydrogenation reaction to produce HFC-143.

a. Single-Reactor Method—Reactor Temperature

In the single-reactor method, the reaction temperature inside reactor 106 may be as low as about 200° C., about 205° C., about 210° C., about 215° C., about 220° C., about 225° C., about 230° C., about 235° C., about 240° C., about 245° C., about 250° C., about 255° C., or as high as about 260° C., about 265° C., about 270° C., about 275° C., about 280° C., about 285° C., about 290° C., about 295° C., about 300° C., about 305° C., about 310° C., about 315° C., about 320° C., about 325° C., about 350° C., or within any range encompassed by any two of the foregoing values as endpoints. For example, the reaction temperature may be from about 200° C. to about 350° C., from about 200° C. to about 325° C., from about 210° C. to about 325° C., from about 220° C. to about 325° C., from about 230° C. to about 325° C., from about 240° C. to about 325° C., from about 250° C. to about 325° C., from about 260° C. to about 325° C., from about 270° C. to about 325° C., or from about 280° C. to about 320° C. Specific examples of additional suitable ranges are set forth below in Table 9.

TABLE 9
Reactor Temperature Range for Single-Reactor Method using Catalyst
(Pd or Pt or Rh) on Supports (Activated Carbon, alpha(α)-Al₂O₃,
theta(θ)-Al₂O₃, delta(δ)-Al₂O₃, or gamma(γ)-Al₂O₃) -
Each Catalyst/Support Combination (Each Row) in Table 1 -
Step (i) Hydrogenation Reaction

| From (° C.) | To (° C.) |
|---|---|
| 200 | 350 |
| 200 | 325 |
| 200 | 320 |
| 220 | 350 |
| 220 | 325 |
| 220 | 320 |
| 250 | 350 |
| 250 | 325 |
| 250 | 320 |
| 260 | 350 |
| 260 | 325 |
| 260 | 320 |
| 270 | 350 |
| 270 | 325 |
| 270 | 320 |
| 280 | 350 |
| 280 | 325 |
| 280 | 320 |
| 200 | 220 |
| 220 | 240 |
| 240 | 260 |
| 260 | 280 |
| 280 | 300 |
| 300 | 325 |
| 325 | 350 |

b. Single-Reactor Method—Reaction Conditions

Each catalyst/support combination (each row) in Table 1 may be used in the reactor of the single-reactor method for carrying out the hydrogenation reaction of Step (i). The catalyst/support combination used in the reactor of a single-reactor method may preferably be palladium metal on an alpha alumina support, or more preferably palladium metal on a carbon support (Pd/C). The catalyst loading may be relatively high to encourage conversion of the less reactive intermediates. For example, the Pd loading on a carbon or alpha lumina support may be from about 0.1 wt. % to about 10 wt. % palladium metal, such as about 0.1 wt. %, about 0.2 wt. %, about 0.5 wt. %, about 2 wt. %, about 3 wt. %, 4 wt. %, about 5 wt. %, about 6 wt. %, about 7 wt. %, about 8 wt. %, about 9 wt. %, or about 10 wt. % palladium metal, or within any range encompassed by any two of the foregoing values as endpoints, based on a combined weight of the palladium metal and the carbon or alpha alumina support. A summary of the preferred catalyst and support, loading, and temperatures as discussed above are summarized in Table 10 below.

TABLE 10
Summary of Preferred Catalysts (Palladium, Platinum,
Rhodium) on Supports (Carbon, Alpha Alumina) for Single-
Reactor Method - Step (i) Hydrogenation Reaction

| | | Loading | Temperature |
|---|---|---|---|
| Catalyst | Support | (wt. %) | (° C.) |
| Pd | Activated carbon | 0.5-10 | 200-350 |
| Pd | Activated carbon | 0.5-10 | 230-290 |
| Pd | Activated carbon | 0.5-10 | 250-280 |
| Pd | Activated carbon | 0.5-10 | 250-350 |
| Pd | Activated carbon | 1-5 | 200-350 |
| Pd | Activated carbon | 1-5 | 230-290 |
| Pd | Activated carbon | 1-5 | 250-280 |
| Pd | Activated carbon | 1-5 | 250-350 |
| Pd | alpha(α)-Al₂O₃ | 0.1-10 | 200-350 |
| Pd | alpha(α)-Al₂O₃ | 0.1-10 | 230-290 |
| Pd | alpha(α)-Al₂O₃ | 0.1-10 | 250-280 |
| Pd | alpha(α)-Al₂O₃ | 0.1-10 | 250-350 |
| Pd | alpha(α)-Al₂O₃ | 0.2-5 | 200-350 |
| Pd | alpha(α)-Al₂O₃ | 0.2-5 | 230-290 |
| Pd | alpha(α)-Al₂O₃ | 0.2-5 | 250-280 |
| Pd | alpha(α)-Al₂O₃ | 0.2-5 | 250-350 |
| Pt | Activated carbon | 0.5-10 | 200-350 |
| Pt | Activated carbon | 0.5-10 | 230-290 |
| Pt | Activated carbon | 0.5-10 | 250-280 |
| Pt | Activated carbon | 0.5-10 | 250-350 |
| Pt | Activated carbon | 1-5 | 200-350 |
| Pt | Activated carbon | 1-5 | 230-290 |
| Pt | Activated carbon | 1-5 | 250-280 |
| Pt | Activated carbon | 1-5 | 250-350 |
| Pt | alpha(α)-Al₂O₃ | 0.1-10 | 200-350 |
| Pt | alpha(α)-Al₂O₃ | 0.1-10 | 230-290 |
| Pt | alpha(α)-Al₂O₃ | 0.1-10 | 250-280 |
| Pt | alpha(α)-Al₂O₃ | 0.1-10 | 250-350 |
| Pt | alpha(α)-Al₂O₃ | 0.2-5 | 200-350 |
| Pt | alpha(α)-Al₂O₃ | 0.2-5 | 230-290 |
| Pt | alpha(α)-Al₂O₃ | 0.2-5 | 250-280 |
| Pt | alpha(α)-Al₂O₃ | 0.2-5 | 250-350 |
| Rh | Activated carbon | 0.5-10 | 200-350 |
| Rh | Activated carbon | 0.5-10 | 230-290 |
| Rh | Activated carbon | 0.5-10 | 250-280 |
| Rh | Activated carbon | 0.5-10 | 250-350 |
| Rh | Activated carbon | 1-5 | 200-350 |
| Rh | Activated carbon | 1-5 | 230-290 |
| Rh | Activated carbon | 1-5 | 250-280 |
| Rh | Activated carbon | 1-5 | 250-350 |
| Rh | alpha(α)-Al₂O₃ | 0.1-10 | 200-350 |
| Rh | alpha(α)-Al₂O₃ | 0.1-10 | 230-290 |
| Rh | alpha(α)-Al₂O₃ | 0.1-10 | 250-280 |
| Rh | alpha(α)-Al₂O₃ | 0.1-10 | 250-350 |
| Rh | alpha(α)-Al₂O₃ | 0.2-5 | 200-350 |
| Rh | alpha(α)-Al₂O₃ | 0.2-5 | 230-290 |
| Rh | alpha(α)-Al₂O₃ | 0.2-5 | 250-280 |
| Rh | alpha(α)-Al₂O₃ | 0.2-5 | 250-350 |

Each catalyst/support combination (each row) in Table 1 used in the reactor of the single-reactor method for carrying out the hydrogenation reaction of Step (i) may be pretreated or activated by a variety of methods to improve its performance and effectiveness in the reaction. For example, the catalyst may be dried at elevated temperatures, as low as about 200° C., about 250° C., about 300° C., about 350° C., about 360° C., about 370° C., or as high as about 380° C., about 390° C., about 400° C., about 450° C., about 500° C., about 600° C., about 700° C., or within any range encompassed by two of the foregoing values as endpoints, such as from about 200° C. to about 700° C., from about 250° C. to about 600° C., from about 300° C. to about 500° C., from about 350° C. to about 450° C., from about 360° C., to about 390° C., or from about 370° C. to about 380° C. The catalyst may be dried at a temperature of from about 200° C. to about 700° C., preferably from about 200° C. to about 500° C., most preferably from about 200° C. to about 300° C.

As part of the catalyst pretreatment, each catalyst/support combination (each row) in Table 1 used in the reactor of the single-reactor method for carrying out the hydrogenation reaction of Step (i) may be exposed to an inert gas such as N₂. The pretreatment process may take as low as about 1 hour, about 2 hours, about 3 hours, or as high about 4 hours, about 5 hours, about 6 hours, about 10 hours, about 20 hours, or within any range encompassed by two of the foregoing values as endpoints, such as from about 1 hour to about 20 hours, from about 2 hours to about 10 hours, from about 3 hours to about 6 hours, or about 4 hours to about 5 hours. The catalyst may be exposed to an inert gas such as N₂ for from about 1 hour to about 20 hours, preferably from about 1 hour to about 10 hours, most preferably, from about 1 hour to about 3 hours.

In one example, the hydrogenation reaction of Step (i) may be carried out in the reactor 106 of a single-reactor method using palladium metal catalyst on a catalyst support (Pd/C) with the Pd loading from about 1 wt. % to about 5 wt. % palladium metal based on a combined weight of the palladium metal and the carbon support, at a temperature from about 200° C. to about 350° C.

In another example, the hydrogenation reaction of Step (i) may be carried out in the reactor 106 of a single-reactor method using palladium metal catalyst (Pd) on a catalyst support (Activated Carbon) with the Pd loading from about 2 wt. % to about 4 wt. % palladium metal based on a combined weight of the palladium metal and the carbon support, at a temperature from about 200° C. to about 325° C.

In another example, the hydrogenation reaction of Step (i) may be carried out in the reactor 106 of a single-reactor method using palladium metal catalyst on a catalyst support (Pd/C) with the Pd loading at about 4 wt. % palladium metal based on a combined weight of the palladium metal and the carbon support, at a temperature from about 250° C. to about 350° C.

In one example, the hydrogenation reaction of Step (i) may be carried out in the reactor 106 of a single-reactor method using platinum metal catalyst on a catalyst support (Pt/C) with the Pt loading from about 1 wt. % to about 5 wt. % platinum metal based on a combined weight of the platinum metal and the carbon support, at a temperature from about 200° C. to about 350° C.

In another example, the hydrogenation reaction of Step (i) may be carried out in the reactor 106 of a single-reactor method using platinum metal catalyst (Pt) on a catalyst support (Activated Carbon) with the Pt loading from about 2 wt. % to about 4 wt. % platinum metal based on a combined weight of the platinum metal and the carbon support, at a temperature from about 200° C. to about 325° C.

In another example, the hydrogenation reaction of Step (i) may be carried out in the reactor 106 of a single-reactor method using platinum metal catalyst on a catalyst support (Pt/C) with the Pt loading at about 4 wt. % platinum metal based on a combined weight of the platinum metal and the carbon support, at a temperature from about 250° C. to about 350° C.

In one example, the hydrogenation reaction of Step (i) may be carried out in the reactor 106 of a single-reactor method using rhodium metal catalyst on a catalyst support (Rh/C) with the Rh loading from about 1 wt. % to about 5 wt. % rhodium metal based on a combined weight of the rhodium metal and the carbon support, at a temperature from about 200° C. to about 350° C.

In another example, the hydrogenation reaction of Step (i) may be carried out in the reactor 106 of a single-reactor method using rhodium metal catalyst (Rh) on a catalyst support (Activated Carbon) with the Rh loading from about 2 wt. % to about 4 wt. % rhodium metal based on a combined weight of the rhodium metal and the carbon support, at a temperature from about 200° C. to about 325° C.

In another example, the hydrogenation reaction of Step (i) may be carried out in the reactor 106 of a single-reactor method using rhodium metal catalyst on a catalyst support (Rh/C) with the Rh loading at about 4 wt. % rhodium metal based on a combined weight of the rhodium metal and the carbon support, at a temperature from about 250° C. to about 350° C.

c. Single-Reactor Method—Product Stream from Reactor

Referring again to FIG. 1, the hydrogenation reaction of Step (i) inside reactor 106 for a single-reactor method produces a product stream 108 (before any post processing steps) comprising the target product 1,1,2-trifluoroethane (HFC-143), the recyclable intermediates such as 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a), unreacted starting materials such as hydrogen (H₂) and 1,1,2-trichloro-1,2,2-trifluoroethane (CFC-113), and other undesired byproducts such as ethane (HC-170), 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), chloroethane (HCC-160), 1,1,1-trifluoroethane (HFC-143a), hydrogen chloride (HCl), and hydrogen fluoride (HF).

As discussed below, the product stream 108 from reactor 106 may be subject to one or more post processing steps downstream of reactor 106.

d. Single-Reactor Method—Post Processing

For a hydrogenation reaction of Step (i) carried out using each catalyst/support combination (each row) in Table 1 in a single-reactor method, the product stream 108 from reactor 106 is subsequently fed into a plurality of distillation columns to remove undesired byproducts of the hydrogenation reaction and other impurities. Each distillation column is operated at pressures ranging from 0 to 300 psig. Temperatures of the distillation columns will be determined by the selected pressure.

The product stream 108 from reactor 106 is fed through the first distillation column 110 to recover unreacted hydrogen (H₂). From distillation column 110, overhead stream 112 comprising hydrogen (H₂) may be removed or, alternatively, conveyed back to reactor 106 via stream 114, and bottom stream 116 comprising the product mixture including 1,1,2-trifluoroethane (HFC-143) is conveyed to the second distillation column 118.

The second distillation column 118 is configured to remove hydrogen chloride (HCl) and ethane (HC-170) from the product mixture in the bottom stream 116 from the first distillation column 110. An overhead stream 120 from distillation column 118 includes hydrogen chloride (HCl) and ethane (HC-170) and bottom stream 122 including 1,1,2-trifluoroethane (HFC-143) which is conveyed to scrubber 124 to remove hydrogen fluoride (HF). Stream 126 exits the scrubber 124, goes through an optional dryer column (not shown), and then enters the third distillation column 128 which is configured to remove low boiling point compounds such as 1,1-difluoroethane (HFC-152a), 1-chloro-1,2-difluoroethane (HCFC-142b), trifluoroethylene (HFO-1123), and 1,1,1-trifluoroethane (HFC-143a). Advantageously, the undesired byproducts HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), and/or chloroethane (HCC-160) may be converted to ethane, R-152, and other low boiling molecules and therefore will be easier to remove via distillation using the distillation column 128.

Overhead stream 130 exits the third distillation column 128 and includes byproduct components such as 1,1-difluoroethane (HFC-152a), HCFC-142 isomers (e.g., 1-chloro-1,2-difluoroethane (HCFC-142a) and/or 1-chloro-2,2-difluoroethane (HCFC-142)), and/or 1,1,1-trifluoroethane (HFC-143a).

Bottom stream 132 including 1,1,2-trifluoroethane (HFC-143) is fed into the fourth distillation column 134, from which an overhead stream 136 comprising the desired product 1,1,2-trifluoroethane (HFC-143) is taken.

e. Single-Reactor Method—Desired Product

For a hydrogenation reaction of Step (i) carried out using each catalyst/support combination (each row) in Table 1 in a single-reactor method, the amount or purity of 1,1,2-trifluoroethane (HFC-143) in overhead stream 136 from column 134 may be at least 88 mol %, at least 89 mol %, at least 90 mol %, or at least 91 mol %, and for each of the foregoing, less than or equal to 100 mol %, for example, based on total moles of organic components in the composition.

For a hydrogenation reaction of Step (i) carried out using each catalyst/support combination (each row) in Table 1 in a single-reactor method, the amount of 1-chloro-1,1,2-trifluoroethane (HCFC-133b) in overhead stream 136 may be less than 2000 ppm, less than 1000 ppm, less than 500 ppm, or less than 250 ppm, for example, based on total moles of organic components in the composition.

f. Single-Reactor Method—Recycling of Desired Intermediates

For a hydrogenation reaction of Step (i) carried out using each catalyst/support combination (each row) in Table 1 in a single-reactor method, a bottom recycle stream 138 from the fourth distillation column 134 comprising desired intermediates such as 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a) may be recycled back into reactor 106 via inlet stream 104 so that the foregoing molecules may be further reacted in a continuous manner according to the present single reactor method.

g. Single-Reactor Method—Product Composition

For a hydrogenation reaction of Step (i) carried out using each catalyst/support combination (each row) in Table 1 using a single-reactor method, referring to FIG. 1, the amount of 1,1,2-trifluoroethane (HFC-143) in the product composition in stream 108 from the reactor 106 may be at least 40 mol %, at least 60 mol %, or at least 70 mol %, and for each of the foregoing, less than or equal to 100 mol %, for example, based on total moles of organic components of the composition.

For a hydrogenation reaction of Step (i) carried out using each catalyst/support combination (each row) in Table 1 using a single-reactor method, referring to FIG. 1, the amount of 1-chloro-1,1,2-trifluoroethane (HCFC-133b) in the product composition in stream 108 from the reactor 106, if present, may be greater than or equal to 0.5% and yet less than 40 mol %, less than 30 mol %, less than 25 mol %, or less than 20 mol %, for example, based on total moles of organic components of the composition.

For a hydrogenation reaction of Step (i) carried out using each catalyst/support combination (each row) in Table 1 using a single-reactor method, referring to FIG. 1, the amount of 1-chloro-1,2,2-trifluoroethane (HCFC-133) in the product composition in stream 108 from the reactor 106, if present, may be greater than or equal to 0.1 mol % and yet less than 10 mol %, less than 5 mol %, or less than 1 mol %, for example, based on total moles of organic components of the composition.

For a hydrogenation reaction of Step (i) carried out using each catalyst/support combination (each row) in Table 1 using a single-reactor method, referring to FIG. 1, the amount of 1,2-dichloro-1,1,2-trifluoroethane (HCFC-123a) in the product composition in stream 108 from the reactor 106, if present, may be greater than or equal to 0.1 mol % and yet less than 15 mol %, less than 5 mol %, or less than 0.5 mol %, for example, based on total moles of organic components of the composition.

For a hydrogenation reaction of Step (i) carried out using each catalyst/support combination (each row) in Table 1 using a single-reactor method, referring to FIG. 1, the total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) in the product composition in stream 108 from the reactor 106 may be at least 80 mol %, at least 85 mol %, or at least 90 mol %, and for each of the foregoing, less than or equal to 100 mol %, for example, based on total moles of organic components of the composition.

For example, for a hydrogenation reaction of Step (i) carried out using each catalyst/support combination (each row) in Table 1 using a single-reactor method, referring to FIG. 1, in the product composition in stream 108 from the reactor 106, the amount of 1,1,2-trifluoroethane (HFC-143) may be at least 45 mol % and less than or equal to 99.3 mol %, the amount of 1-chloro-1,1,2-trifluoroethane (HCFC-133b) may be at least 0.5 mol % and less than or equal to 40 mol %, the amount of 1-chloro-1,2,2-trifluoroethane (HCFC-133) may be at least 0.1 mol % and less than or equal to 5 mol %, and the amount of 1,2-dichloro-1,1,2-trifluoroethane (HCFC-123a) may be at least 0.1 mol % and less than or equal to 10 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 80 mol % and less than or equal to 100% of the total moles of organic components of the product composition in stream 108 from the reactor 106, while the combined amount of other components including undesired byproducts (e.g., HFC-143a, HC-170, HCC-160, HFC-152a, and HCFC-142 isomers) may be greater than or equal to 0 mol % and less than 20 mol % of the total moles of organic components of the product composition in stream 108 from the reactor 106.

For example, for a hydrogenation reaction of Step (i) carried out using each catalyst/support combination (each row) in Table 1 using a single-reactor method, referring to FIG. 1, in the product composition in stream 108 from the reactor 106, the amount of 1,1,2-trifluoroethane (HFC-143) may be at least 65 mol % and less than or equal to 99.3 mol %, the amount of 1-chloro-1,1,2-trifluoroethane (HCFC-133b) may be at least 0.5 mol % and less than or equal to 25 mol %, the amount of 1-chloro-1,2,2-trifluoroethane (HCFC-133) may be at least 0.1 mol % and less than or equal to 5 mol %, and the amount of 1,2-dichloro-1,1,2-trifluoroethane (HCFC-123a) may be at least 0.1 mol % and less than or equal to 5 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 85 mol % and less than or equal to 100% of the total moles of organic components of the product composition in stream 108 from the reactor 106, while the combined amount of other components including undesired byproducts (e.g., HFC-143a, HC-170, HCC-160, HFC-152a, and HCFC-142 isomers) may be greater than or equal to 0 mol % and less than 15 mol % of the total moles of organic components of the product composition in stream 108 from the reactor 106.

For example, for a hydrogenation reaction of Step (i) carried out using each catalyst/support combination (each row) in Table 1 using a single-reactor method, referring to FIG. 1, in the product composition in stream 108 from the reactor 106, the amount of 1,1,2-trifluoroethane (HFC-143) may be at least 78.5 mol % and less than or equal to 99.3 mol %, the amount of 1-chloro-1,1,2-trifluoroethane (HCFC-133b) may be at least 0.5 mol % and less than or equal to 20 mol %, the amount of 1-chloro-1,2,2-trifluoroethane (HCFC-133) may be at least 0.1 mol % and less than or equal to 1 mol %, and the amount of 1,2-dichloro-1,1,2-trifluoroethane (HCFC-123a) may be at least 0.1 mol % and less than or equal to 0.5 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 90 mol % and less than or equal to 100% of the total moles of organic components of the product composition in stream 108 from the reactor 106, while the combined amount of other components including undesired byproducts (e.g., HFC-143a, HC-170, HCC-160, HFC-152a, and HCFC-142 isomers) may be greater than or equal to 0 mol % and less than 10 mol % of the total moles of organic components of the product composition in stream 108 from the reactor 106.

h. Single-Reactor Method—Recycle Stream Composition

For a hydrogenation reaction of Step (i) carried out using each catalyst/support combination (each row) in Table 1 using a single-reactor method, referring to FIG. 1, the amount of 1,1,2-trifluoroethane (HFC-143) in the recycle stream 138 to the reactor 106, if present, may be less than 50 mol %, less than 40 mol %, or less than 3 mol %, and for each of the foregoing, greater than or equal to 0.01 mol %, for example, based on total moles of organic components of the composition.

For a hydrogenation reaction of Step (i) carried out using each catalyst/support combination (each row) in Table 1 using a single-reactor method, referring to FIG. 1, the amount of 1-chloro-1,1,2-trifluoroethane (HCFC-133b) in the recycle stream 138 to the reactor 106 may be greater than 20 mol %, greater than 40 mol %, or greater than 60 mol %, and for each of the foregoing, less than or equal to 100 mol %, for example, based on total moles of organic components of the composition.

For a hydrogenation reaction of Step (i) carried out using each catalyst/support combination (each row) in Table 1 using a single-reactor method, referring to FIG. 1, the amount of 1-chloro-1,2,2-trifluoroethane (HCFC-133) in the recycle stream 138 to the reactor 106, if present, may be less than 10 mol %, less than 5 mol %, or less than 1 mol %, and for each of the foregoing, greater than or equal to 0.01 mol %, for example, based on total moles of organic components of the composition.

For a hydrogenation reaction of Step (i) carried out using each catalyst/support combination (each row) in Table 1 using a single-reactor method, referring to FIG. 1, the amount of 1,2-dichloro-1,1,2-trifluoroethane (HCFC-123a) in the recycle stream 138 to the reactor 106, if present, may be less than 20 mol %, less than 15 mol %, or less than 9 mol %, and for each of the foregoing, greater than or equal to 0.01 mol %, for example, based on total moles of organic components of the composition.

For a hydrogenation reaction of Step (i) carried out using each catalyst/support combination (each row) in Table 1 using a single-reactor method, referring to FIG. 1, the total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) in the recycle stream 138 to the reactor 106 may be at least 80 mol %, at least 90 mol %, or at least 95 mol %, and for each of the foregoing, less than or equal to 100 mol %, for example, based on total moles of organic components of the composition.

For example, for a hydrogenation reaction of Step (i) carried out using each catalyst/support combination (each row) in Table 1 using a single-reactor method, referring to FIG. 1, in the recycle stream 138 to the reactor 106, the amount of 1,1,2-trifluoroethane (HFC-143) may be less than 50 mol % and greater than or equal to 0 mol %, the amount of 1-chloro-1,1,2-trifluoroethane (HCFC-133b) may be at least 20 mol % and less than or equal to 100 mol %, the amount of 1-chloro-1,2,2-trifluoroethane (HCFC-133) may be less than 10 mol % and greater than or equal to 0 mol %, the amount of 1,2-dichloro-1,1,2-trifluoroethane (HCFC-123a) may be less than 20 mol % and greater than or equal to 0 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 80 mol % and less than or equal to 100% of the total moles of organic components of the product composition in the recycle stream 138 to the reactor 106, while the combined amount of other components including undesired byproducts (e.g., HFC-143a, HC-170, HCC-160, HFC-152a, and HCFC-142 isomers) may be greater than or equal to 0 mol % and less than 20 mol % of the total moles of organic components of the product composition in the recycle stream 138 to the reactor 106.

For example, for a hydrogenation reaction of Step (i) carried out using each catalyst/support combination (each row) in Table 1 using a single-reactor method, referring to FIG. 1, in the recycle stream 138 to the reactor 106, the amount of 1,1,2-trifluoroethane (HFC-143) may be less than 40 mol % and greater than or equal to 0 mol %, the amount of 1-chloro-1,1,2-trifluoroethane (HCFC-133b) may be greater than 40 mol % and less than or equal to 100 mol %, the amount of 1-chloro-1,2,2-trifluoroethane (HCFC-133) may be less than 5 mol % and greater than or equal to 0 mol %, the amount of 1,2-dichloro-1,1,2-trifluoroethane (HCFC-123a) may be less than 15 mol % and greater than or equal to 0 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 90 mol % and less than or equal to 100% of the total moles of organic components of the product composition in the recycle stream 138 to the reactor 106, while the combined amount of other components including undesired byproducts (e.g., HFC-143a, HC-170, HCC-160, HFC-152a, and HCFC-142 isomers) may be greater than or equal to 0 mol % and less than 10 mol % of the total moles of organic components of the product composition in the recycle stream 138 to the reactor 106.

For example, for a hydrogenation reaction of Step (i) carried out using each catalyst/support combination (each row) in Table 1 using a single-reactor method, referring to FIG. 1, in the recycle stream 138 to the reactor 106, the amount of 1,1,2-trifluoroethane (HFC-143) may be less than 30 mol % and greater than or equal to 0 mol %, the amount of 1-chloro-1,1,2-trifluoroethane (HCFC-133b) may be greater than 60 mol % and less than or equal to 100 mol %, the amount of 1-chloro-1,2,2-trifluoroethane (HCFC-133) may be less than 1 mol % and greater than or equal to 0 mol %, the amount of 1,2-dichloro-1,1,2-trifluoroethane (HCFC-123a) may be less than 9 mol % and greater than or equal to 0 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 95 mol % and less than or equal to 100% of the total moles of organic components of the product composition in the recycle stream 138 to the reactor 106, while the combined amount of other components including undesired byproducts (e.g., HFC-143a, HC-170, HCC-160, HFC-152a, and HCFC-142 isomers) may be greater than or equal to 0 mol % and less than 5 mol % of the total moles of organic components of the product composition in the recycle stream 138 to the reactor 106.

ii. Two-Reactor Method

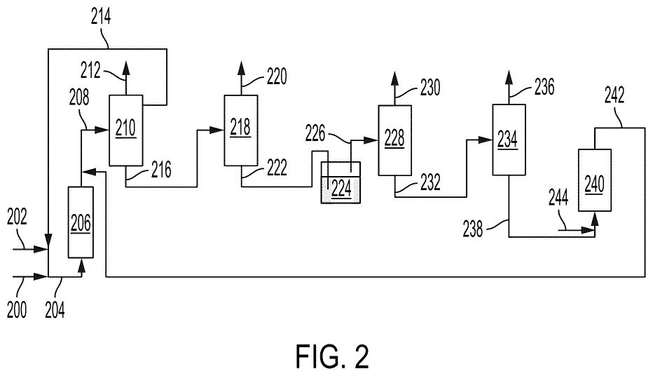

In a two-reactor method used for carrying out a hydrogenation reaction of Step (i) using each catalyst/support combination (each row) in Table 1, the overall reaction method is as described above in connection with FIG. 1, though in connection with certain aspects described below in connection with FIG. 2, the reaction conditions may be selectively tailored to maximize the production of the desired product and intermediates, while minimizing the production of undesired byproducts using, for example, two reactors. In the first reactor, the hydrogenation may proceed at a relatively lower temperature, followed by an optional distillation and acid removal. Then, the products from the first reactor are reacted again in a second reactor in the two-reactor method.

For example, referring to FIG. 2, the first reactor 206 is fed by stream 200 comprising 1,1,2-trichloro-1,2,2-trifluoroethane (CFC-113) and by stream 202 comprising hydrogen (H₂). In the first reactor 206, CFC-113 may be hydrogenated to produce a first product composition in stream 208 comprising the target product 1,1,2-trifluoroethane (HFC-143), the recyclable intermediates such as 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a), unreacted starting materials such as hydrogen (H₂) and 1,1,2-trichloro-1,2,2-trifluoroethane (CFC-113), and other undesired byproducts such as ethane (HC-170), 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), chloroethane (HCC-160), 1,1,1-trifluoroethane (HFC-143a), hydrogen chloride (HCl), and hydrogen fluoride (HF).

a. Two-Reactor Method—First Reactor Temperature

Referring again to FIG. 2, in the first reactor 206 of a two-reactor method, the temperature may be as low as about 200° C., about 205° C., about 210° C., about 215° C., about 220° C., about 225° C., about 230° C., about 235° C., about 240° C., about 245° C., or as high as about 255° C., about 260° C., about 265° C., about 270° C., about 275° C., about 280° C., about 285° C., about 290° C., about 295° C., about 300° C., or within any range encompassed by any two of the foregoing values as endpoints. For example, the reaction temperature may be from about 200° C. to about 300° C., about 205° C. to about 295° C., from about 210° C. to about 290° C., from about 215° C. to about 285° C., from about 220° to about 280° C., from about 225° C. to about 275° C., from about 230° C. to about 270° C., from about 235° C. to about 265° C., from about 240° C. to about 260° C., or about 245° C. to about 255° C.

b. Two-Reactor Method—First Reactor Catalyst

Each catalyst/support combination (each row) in Table 1 may be used in the first reactor 206 of the two-reactor method for carrying out the hydrogenation reaction of Step (i). In one example, the hydrogenation reaction of Step (i) may be carried out in a first reactor 206 of the two-reactor method using palladium metal catalyst on a carbon catalyst support (Pd/C) with the Pd loading from about 1 wt. % to about 5 wt. % palladium metal based on a combined weight of the palladium metal and the carbon support, at a temperature from about 200° C. to about 400° C.

In another example, the hydrogenation reaction of step (i) may be carried out in the first reactor 206 of the two-reactor method using palladium metal catalyst on a carbon catalyst support (Pd/C) with the Pd loading from about 2 wt. % to about 4 wt. % palladium metal based on a combined weight of the palladium metal and the carbon support, at a temperature from about 220° C. to about 400° C.

In another example, the hydrogenation reaction of step (i) may be carried out in the first reactor 206 of the two-reactor method using palladium metal catalyst on a carbon catalyst support (Pd/C) with the Pd loading at about 4 wt. % palladium metal based on a combined weight of the palladium metal and the carbon support, at a temperature from about 250° C. to about 350° C.

In another example, the hydrogenation reaction of step (i) may be carried out in the first reactor 206 of the two-reactor method using palladium metal catalyst on an alpha alumina catalyst support (Pd/alpha(α)-Al₂O₃) with the Pd loading from about 0.1 wt. % to about 10 wt. % palladium metal based on a combined weight of the palladium metal and the alumina support, at a temperature from about 150° C. to about 350° C.

In another example, the hydrogenation reaction of step (i) may be carried out in the first reactor 206 of the two-reactor method using palladium metal catalyst on an alpha alumina catalyst support (Pd/alpha(α)-Al₂O₃) with the Pd loading from about 0.2 wt. % to about 5 wt. % palladium metal based on a combined weight of the palladium metal and the alumina support, at a temperature from about 150° C. to about 350° C.

A summary of the preferred catalyst and support, loading, and temperatures as discussed above in connection with the hydrogenation reaction of step (i) carried out in the first reactor 206 of the two-reactor method are summarized in Table 11 below.

TABLE 11
Summary of Preferred Catalyst (Pd, Pt, Rh) on Support
(carbon, alpha alumina) - First Reactor of Two-Reactor
Method - Step (i) Hydrogenation Reaction

| | | Loading | Temperature |
|---|---|---|---|
| Catalyst | Support | (wt. %) | (° C.) |
| Pd | Activated carbon | 0.5-10 | 200-400 |
| Pd | Activated carbon | 0.5-10 | 220-400 |
| Pd | Activated carbon | 0.5-10 | 240-350 |
| Pd | Activated carbon | 0.5-10 | 250-300 |
| Pd | Activated carbon | 1-5 | 200-400 |
| Pd | Activated carbon | 1-5 | 220-400 |
| Pd | Activated carbon | 1-5 | 240-350 |
| Pd | Activated carbon | 1-5 | 250-300 |
| Pd | Activated carbon | 2-4 | 200-400 |
| Pd | Activated carbon | 2-4 | 220-400 |
| Pd | Activated carbon | 2-4 | 240-350 |
| Pd | Activated carbon | 2-4 | 250-300 |
| Pd | alpha(α)-Al₂O₃ | 0.1-10 | 150-300 |
| Pd | alpha(α)-Al₂O₃ | 0.1-10 | 150-275 |
| Pd | alpha(α)-Al₂O₃ | 0.2-5 | 150-250 |
| Pd | alpha(α)-Al₂O₃ | 0.2-5 | 150-200 |
| Pt | Activated carbon | 0.5-10 | 200-400 |
| Pt | Activated carbon | 0.5-10 | 220-400 |
| Pt | Activated carbon | 0.5-10 | 240-350 |
| Pt | Activated carbon | 0.5-10 | 250-300 |
| Pt | Activated carbon | 1-5 | 200-400 |
| Pt | Activated carbon | 1-5 | 220-400 |
| Pt | Activated carbon | 1-5 | 240-350 |
| Pt | Activated carbon | 1-5 | 250-300 |
| Pt | Activated carbon | 2-4 | 200-400 |
| Pt | Activated carbon | 2-4 | 220-400 |
| Pt | Activated carbon | 2-4 | 240-350 |
| Pt | Activated carbon | 2-4 | 250-300 |
| Pt | alpha(α)-Al₂O₃ | 0.1-10 | 150-300 |
| Pt | alpha(α)-Al₂O₃ | 0.1-10 | 150-275 |
| Pt | alpha(α)-Al₂O₃ | 0.2-5 | 150-250 |
| Pt | alpha(α)-Al₂O₃ | 0.2-5 | 150-200 |
| Rh | Activated carbon | 0.5-10 | 200-400 |
| Rh | Activated carbon | 0.5-10 | 220-400 |
| Rh | Activated carbon | 0.5-10 | 240-350 |
| Rh | Activated carbon | 0.5-10 | 250-300 |
| Rh | Activated carbon | 1-5 | 200-400 |
| Rh | Activated carbon | 1-5 | 220-400 |
| Rh | Activated carbon | 1-5 | 240-350 |
| Rh | Activated carbon | 1-5 | 250-300 |
| Rh | Activated carbon | 2-4 | 200-400 |
| Rh | Activated carbon | 2-4 | 220-400 |
| Rh | Activated carbon | 2-4 | 240-350 |
| Rh | Activated carbon | 2-4 | 250-300 |
| Rh | alpha(α)-Al₂O₃ | 0.1-10 | 150-300 |
| Rh | alpha(α)-Al₂O₃ | 0.1-10 | 150-275 |
| Rh | alpha(α)-Al₂O₃ | 0.2-5 | 150-250 |
| Rh | alpha(α)-Al₂O₃ | 0.2-5 | 150-200 |

c. Two-Reactor Method—First Reactor Product Stream Composition

For a hydrogenation reaction of Step (i) carried out using each catalyst/support combination (each row) in Table 1 using a two-reactor method, referring to FIG. 2, the amount of 1,1,2-trifluoroethane (HFC-143) in the first product stream 208 from the first reactor 206 of the two-reactor method may be at least 40 mol %, at least 60 mol %, or at least 70 mol %, and for each of the foregoing, less than or equal to 100 mol %, for example, based on total moles of organic components of the composition.

For a hydrogenation reaction of Step (i) carried out using each catalyst/support combination (each row) in Table 1 using a two-reactor method, referring to FIG. 2, the amount of 1-chloro-1,1,2-trifluoroethane (HCFC-133b) in the first product stream 208 from the first reactor 206 of the two-reactor method, if present, may be greater than 0.5% and yet less than 40 mol %, less than 30 mol %, less than 25 mol %, or less than 20 mol %, for example, based on total moles of organic components of the composition.

For a hydrogenation reaction of Step (i) carried out using each catalyst/support combination (each row) in Table 1 using a two-reactor method, referring to FIG. 2, the amount of 1-chloro-1,2,2-trifluoroethane (HCFC-133) in the first product stream 208 from the first reactor 206 of the two-reactor method, if present, may be greater than 0.1 mol % and yet less than 10 mol %, less than 5 mol %, or less than 1 mol %, for example, based on total moles of organic components of the composition.

For a hydrogenation reaction of Step (i) carried out using each catalyst/support combination (each row) in Table 1 using a two-reactor method, referring to FIG. 2, the amount of 1,2-dichloro-1,1,2-trifluoroethane (HCFC-123a) in the first product stream 208 from the first reactor 206 of the two-reactor method, if present, may be greater than 0.1 mol % and yet less than 15 mol %, less than 5 mol %, or less than 0.5 mol %, for example, based on total moles of organic components of the composition.

For a hydrogenation reaction of Step (i) carried out using each catalyst/support combination (each row) in Table 1 using a two-reactor method, referring to FIG. 2, the total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) in the first product stream 208 from the first reactor 206 of the two-reactor method may be at least 80 mol %, at least 85 mol %, or at least 90 mol %, and for each of the foregoing, less than or equal to 100 mol %, for example, based on total moles of organic components of the composition.

For example, for a hydrogenation reaction of Step (i) carried out using each catalyst/support combination (each row) in Table 1 using a two-reactor method, referring to FIG. 2, in the first product stream 208 from the first reactor 206 of the two-reactor method, the amount of 1,1,2-trifluoroethane (HFC-143) may be at least 45 mol % and less than or equal to 99.3 mol %, the amount of 1-chloro-1,1,2-trifluoroethane (HCFC-133b) may be at least 0.5 mol % and less than or equal to 40 mol %, the amount of 1-chloro-1,2,2-trifluoroethane (HCFC-133) may be at least 0.1 mol % and less than or equal to 5 mol %, and the amount of 1,2-dichloro-1,1,2-trifluoroethane (HCFC-123a) may be at least 0.1 mol % and less than or equal to 10 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 85 mol % and less than or equal to 100% of the total moles of organic components of the product composition in stream 208 from the first reactor 206, while the combined amount of other components including undesired byproducts (e.g., HFC-143a, HC-170, HCC-160, HFC-152a, and HCFC-142 isomers) may be greater than or equal to 0 mol % and less than 15 mol % of the total moles of organic components of the product composition in stream 208 from the first reactor 206.

For example, for a hydrogenation reaction of Step (i) carried out using each catalyst/support combination (each row) in Table 1 using a two-reactor method, referring to FIG. 2, in the first product stream 208 from the first reactor 206 of the two-reactor method, the amount of 1,1,2-trifluoroethane (HFC-143) may be at least 65 mol % and less than or equal to 99.3 mol %, the amount of 1-chloro-1,1,2-trifluoroethane (HCFC-133b) may be at least 0.5 mol % and less than or equal to 25 mol %, the amount of 1-chloro-1,2,2-trifluoroethane (HCFC-133) may be at least 0.1 mol % and less than or equal to 5 mol %, and the amount of 1,2-dichloro-1,1,2-trifluoroethane (HCFC-123a) may be at least 0.1 mol % and less than or equal to 5 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 90 mol % and less than or equal to 100% of the total moles of organic components of the product composition in stream 208 from the first reactor 206, while the combined amount of other components including undesired byproducts (e.g., HFC-143a, HC-170, HCC-160, HFC-152a, and HCFC-142 isomers) may be greater than or equal to 0 mol % and less than 10 mol % of the total moles of organic components of the product composition in stream 208 from the first reactor 206.

For example, for a hydrogenation reaction of Step (i) carried out using each catalyst/support combination (each row) in Table 1 using a two-reactor method, referring to FIG. 2, in the first product stream 208 from the first reactor 206 of the two-reactor method, the amount of 1,1,2-trifluoroethane (HFC-143) may be at least 78.5 mol % and less than or equal to 99.3 mol %, the amount of 1-chloro-1,1,2-trifluoroethane (HCFC-133b) may be at least 0.5 mol % and less than or equal to 20 mol %, the amount of 1-chloro-1,2,2-trifluoroethane (HCFC-133) may be at least 0.1 mol % and greater than or equal to 1 mol %, and the amount of 1,2-dichloro-1,1,2-trifluoroethane (HCFC-123a) may be greater than 0.1 mol % and less than 0.5 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 95 mol % and less than or equal to 100% of the total moles of organic components of the product composition in stream 208 from the first reactor 206, while the combined amount of other components including undesired byproducts (e.g., HFC-143a, HC-170, HCC-160, HFC-152a, and HCFC-142 isomers) may be greater than or equal to 0 mol % and less than 5 mol % of the total moles of organic components of the product composition in stream 208 from the first reactor 206.

As discussed below, the products from the first reactor 206 may be subject to one or more post processing steps downstream of the first reactor 206 before entering a second reactor 240 of the two-reactor method.

d. Two-Reactor Method—First Reactor Post Processing

Referring again to FIG. 2, before the first product composition in the first product stream 208 from the first reactor 206 is fed into the second reactor 240 in a two-reactor method, the first product stream 208 may optionally be distilled and/or subjected to removal of acids to improve purity and prevent catalyst contamination in any subsequent reactions. Referring again to FIG. 2, the first product stream 208 enters distillation column 210 to remove unreacted hydrogen (H₂) via the top stream 212. Alternatively, from distillation column 210, overhead stream 214 comprising hydrogen (H₂) may be conveyed back to reactor 206.

Bottom stream 216 is then conveyed to the second distillation column 218 to remove hydrogen chloride (HCl) and ethane (HC-170). The second distillation column 218 generates overhead stream 220 comprising hydrogen chloride (HCl) and ethane (HC-170) and bottom stream 222 comprising 1,1,2-trifluoroethane (HFC-143) which is then fed into caustic solution scrubber 224 to remove hydrogen fluoride (HF). Optionally, the overhead 220 stream comprising HCl can be further treated/purified to produce salable HCl product. Optionally the stream 226 can be dried, for example, using solid adsorbent such as 3 A/4 A molecular sieve.

Stream 226, which is essentially acid free, exits caustic solution scrubber 224 and enters the third distillation column 228 to remove low boiling molecules. Distillation column 228 generates overhead stream 230 comprising 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), 1,1,2-trifluoroethene (HFO-1123), and 1,1,1-trifluoroethane (HFC-143a) and bottom stream 232 comprising 1,1,2-trifluoroethane (HFC-143).

Bottom stream 232 is then fed into the fourth distillation column 234. Overhead stream 236 exits distillation column 234 and comprises the desired product 1,1,2-trifluoroethane (HFC-143) which is removed from the system.

e. Two-Reactor Method—Overhead Stream 236 Composition

For a hydrogenation reaction of Step (i) carried out using each catalyst/support combination (each row) in Table 1 using a two-reactor method, referring to FIG. 2, the amount of 1,1,2-trifluoroethane (HFC-143) in overhead stream 236 may be greater than 88 mol %, greater than 89 mol %, greater than 90 mol %, or greater than 91 mol %, and for each of the foregoing, less than or equal to 100 mol %, for example, based on total moles of organic components in the composition.

For a hydrogenation reaction of Step (i) carried out using each catalyst/support combination (each row) in Table 1 using a two-reactor method, referring to FIG. 2, the amount of 1-chloro-1,1,2-trifluoroethane (HCFC-133b) in overhead stream 236, if present, may be less than 2000 ppm, less than 1000 ppm, less than 500 ppm, or less than 250 ppm, and for each of the foregoing, greater than or equal to 0 ppm, for example, based on total moles of organic components in the composition.

f. Two-Reactor Method—Bottom Stream 238 Composition

For a hydrogenation reaction of Step (i) carried out using each catalyst/support combination (each row) in Table 1 using a two-reactor method, referring to FIG. 2, the amount of 1,1,2-trifluoroethane (HFC-143) in the bottom stream 238 from the fourth distillation column 234, if present, may be less than 50 mol %, less than 20 mol %, or less than 10 mol %, and for each of the foregoing, greater than or equal to 0.01 mol %, for example, based on total moles of organic components of the composition.

For a hydrogenation reaction of Step (i) carried out using each catalyst/support combination (each row) in Table 1 using a two-reactor method, referring to FIG. 2, the amount of 1-chloro-1,1,2-trifluoroethane (HCFC-133b) in the bottom stream 238 from the fourth distillation column 234 may be greater than 50 mol %, greater than 60 mol %, or greater than 70 mol %, and for each of the foregoing, less than or equal to 100 mol %, for example, based on total moles of organic components of the composition.

For a hydrogenation reaction of Step (i) carried out using each catalyst/support combination (each row) in Table 1 using a two-reactor method, referring to FIG. 2, the amount of 1-chloro-1,2,2-trifluoroethane (HCFC-133) in the bottom stream 238 from the fourth distillation column 234, if present, may be less than 20 mol %, less than 10 mol %, or less than 1 mol %, and for each of the foregoing, greater than or equal to 0.01 mol %, for example, based on total moles of organic components of the composition.

For a hydrogenation reaction of Step (i) carried out using each catalyst/support combination (each row) in Table 1 using a two-reactor method, referring to FIG. 2, the amount of 1,2-dichloro-1,1,2-trifluoroethane (HCFC-123a) in the bottom stream 238 from the fourth distillation column 234, if present, may less than 30 mol %, less than 20 mol %, or less than 10 mol %, and for each of the foregoing, greater than or equal to 0.01 mol %, for example, based on total moles of organic components of the composition.

For a hydrogenation reaction of Step (i) carried out using each catalyst/support combination (each row) in Table 1 using a two-reactor method, referring to FIG. 2, the total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) in the bottom stream 238 from the fourth distillation column 234 may be at least 80 mol %, at least 90 mol %, or at least 95 mol %, and for each of the foregoing, less than or equal to 100 mol %, for example, based on total moles of organic components of the composition.

For example, for a hydrogenation reaction of Step (i) carried out using each catalyst/support combination (each row) in Table 1 using a two-reactor method, referring to FIG. 2, in the bottom stream 238 from the fourth distillation column 234, the amount of 1,1,2-trifluoroethane (HFC-143) may be less than 50 mol % and greater than or equal to 0 mol %, the amount of 1-chloro-1,1,2-trifluoroethane (HCFC-133b) may be at least 20 mol % and less than or equal to 100 mol %, the amount of 1-chloro-1,2,2-trifluoroethane (HCFC-133) may be less than 10 mol % and greater than or equal to 0 mol %, the amount of 1,2-dichloro-1,1,2-trifluoroethane (HCFC-123a) may be less than 20 mol % and greater than or equal to 0 mol %, based on the combined total moles of the HFC-143, HCFC-133b, HCFC-133, and HCFC-123a in the product composition.

The combined amount of HFC-143, HCFC-133b, HCFC-133, and HCFC-123a in the composition set forth in the preceding paragraph may be at least 85 mol % and less than or equal to 100% of the total moles of organic components of the product composition in the bottom stream 238 from the fourth distillation column 234, while the combined amount of other components including undesired byproducts (e.g., HFC-143a, HC-170, HCC-160, HFC-152a, and HCFC-142 isomers) may be greater than or equal to 0 mol % and less than 15 mol % of the total moles of organic components of the product composition in the bottom stream 238 from the fourth distillation column 234.

For example, for a hydrogenation reaction of Step (i) carried out using each catalyst/support combination (each row) in Table 1 using a two-reactor method, referring to FIG. 2, in the bottom stream 238 from the fourth distillation column 234, the amount of 1,1,2-trifluoroethane (HFC-143) may be less than 40 mol % and greater than or equal to 0 mol %, the amount of 1-chloro-1,1,2-trifluoroethane (HCFC-133b) may be greater than 40 mol % and less than or equal to 100 mol %, the amount of 1-chloro-1,2,2-trifluoroethane (HCFC-133) may be less than 5 mol % and greater than or equal to 0 mol %, the amount of 1,2-dichloro-1,1,2-trifluoroethane (HCFC-123a) may be less than 15 mol % and greater than or equal to 0 mol %, based on the combined total moles of the HFC-143, HCFC-133b, HCFC-133, and HCFC-123a in the product composition.

The combined amount of HFC-143, HCFC-133b, HCFC-133, and HCFC-123a in the composition set forth in the preceding paragraph may be at least 90 mol % and less than or equal to 100% of the total moles of organic components of the product composition in the bottom stream 238 from the fourth distillation column 234, while the combined amount of other components including undesired byproducts (e.g., HFC-143a, HC-170, HCC-160, HFC-152a, and HCFC-142 isomers) may be greater than or equal to 0 mol % and less than 10 mol % of the total moles of organic components of the product composition in the bottom stream 238 from the fourth distillation column 234.

For example, for a hydrogenation reaction of Step (i) carried out using each catalyst/support combination (each row) in Table 1 using a two-reactor method, referring to FIG. 2, in the bottom stream 238 from the fourth distillation column 234, the amount of 1,1,2-trifluoroethane (HFC-143) may be less than 30 mol % and greater than or equal to 0 mol %, the amount of 1-chloro-1,1,2-trifluoroethane (HCFC-133b) may be greater than 60 mol % and less than or equal to 100 mol %, the amount of 1-chloro-1,2,2-trifluoroethane (HCFC-133) may be less than 1 mol % and greater than or equal to 0 mol %, the amount of 1,2-dichloro-1,1,2-trifluoroethane (HCFC-123a) may be less than 9 mol % and greater than or equal to 0 mol %, based on the combined total moles of the HFC-143, HCFC-133b, HCFC-133, and HCFC-123a in the product composition.

The combined amount of HFC-143, HCFC-133b, HCFC-133, and HCFC-123a in the composition set forth in the preceding paragraph may be at least 95 mol % and less than or equal to 100% of the total moles of organic components of the product composition in the bottom stream 238 from the fourth distillation column 234, while the combined amount of other components including undesired byproducts (e.g., HFC-143a, HC-170, HCC-160, HFC-152a, and HCFC-142 isomers) may be greater than or equal to 0 mol % and less than 5 mol % of the total moles of organic components of the product composition in the bottom stream 238 from the fourth distillation column 234.

g. Two-Reactor Method—Second Reactor Hydrogenation

For a hydrogenation reaction of Step (i) carried out using each catalyst/support combination (each row) in Table 1 using a two-reactor method, referring to FIG. 2, the bottom stream 238 from the fourth distillation column 234 is mixed with hydrogen from stream 244 and then sent into the second reactor 240 of the two-reactor method for a second hydrogenation reaction.

Although the bottom stream 238 from the fourth distillation column 234 may be recycled directly back to the reactor 206, it has been discovered that the stream 238 may be further reacted in a second reactor 240 in the two-reactor method to further enhance conversion rate and thus improve selectivity towards desired product and desired intermediates.

h. Two-Reactor Method—Second Reactor Temperature

For a hydrogenation reaction of Step (i) carried out using each catalyst/support combination (each row) in Table 1 using a two-reactor method, referring to FIG. 2, the second reactor 240 of the two-reactor method may be operated at a temperature as low as about 200° C., about 205° C., about 210° C., about 215° C., about 220° C., about 225° C., about 230° C., about 235° C., about 240° C., about 245° C., about 250° C., about 255° C., or as high as about 260° C., about 265° C., about 270° C., about 275° C., about 280° C., about 285° C., about 290° C., about 295° C., about 300° C., about 305° C., about 310° C., about 315° C., about 320° C., about 325° C., or within any range encompassed by any two of the foregoing values as endpoints. For example, the reaction temperature may be from about 200° C. to about 325° C., from about 205° C. to about 320° C., from about 210° C. to about 315° C., from about 215° C. to about 310° C., from about 220° C. to about 305° C., from about 225° C. to about 300° C., from about 230° C. to about 295° C., from about 235° C. to about 290° C., from about 240° C. to about 285° C., from about 245° C. to about 280° C., from about 250° C. to about 275° C., from about 255° C. to about 270° C., or from about 260° C. to about 265° C.

i. Two-Reactor Method—Second Reactor Catalyst

The hydrogenation reaction of Step (i) may be carried out using each catalyst/support combination (each row) in Table 1 in the second reactor of a two-reactor method. In one example, the hydrogenation reaction of Step (i) in the second reactor 240 of the two-reactor method may be carried out using palladium metal catalyst on a catalyst support (Pd/C) with the Pd loading from about 1 wt. % to about 5 wt. % palladium metal based on a combined weight of the palladium metal and the carbon support, at a temperature from about 200° C. to about 450° C.

In another example, the hydrogenation reaction of Step (i) in the second reactor 240 of the two-reactor method may be carried out using palladium metal catalyst on a catalyst support (Pd/C) with the Pd loading from about 2 wt. % to about 4 wt. % palladium metal based on a combined weight of the palladium metal and the carbon support, at a temperature from about 220° C. to about 400° C.

In another example, the hydrogenation reaction of Step (i) in the second reactor 240 of the two-reactor method may be carried out using palladium metal catalyst on a catalyst support (Pd/C) with the Pd loading at about 4 wt. % palladium metal based on a combined weight of the palladium metal and the carbon support, at a temperature from about 250° C. to about 350° C.

In another example, the hydrogenation reaction of Step (i) in the second reactor 240 of the two-reactor method may be carried out using palladium metal catalyst on an alpha alumina catalyst support (Pd/alpha(α)-Al₂O₃) with the Pd loading from about 0.1 wt. % to about 10 wt. % palladium metal based on a combined weight of the palladium metal and the alumina support, at a temperature from about 150° C. to about 350° C.

In another example, the hydrogenation reaction of Step (i) in the second reactor 240 of the two-reactor method may be carried out using palladium metal catalyst on an alpha alumina catalyst support (Pd/alpha(α)-Al₂O₃) with the Pd loading from about 0.2 wt. % to about 5 wt. % palladium metal based on a combined weight of the palladium metal and the alumina support, at a temperature from about 150° C. to about 350° C.

For Step (i) hydrogenation reactions carried out in the second reactor 240 of the two-reactor method, the catalyst may be palladium metal on a carbon support (Pd/C). The catalyst loading may be relatively high to encourage conversion of the less reactive intermediates. For example, the Pd loading may be from about 1 wt. % to about 5 wt. % palladium metal, such as about 1 wt. %, about 2 wt. %, about 3 wt. %, about 4 wt. %, or about 5 wt. % palladium metal, based on a combined weight of the palladium metal and the carbon support.

For Step (i) hydrogenation reactions carried out in the second reactor 240 of the two-reactor method, the catalyst may be palladium metal on an alpha alumina support (Pd/alpha(α)-Al₂O₃). The Pd catalyst loading may be from about 0.1 wt. % to about 10 wt. % palladium metal, or from about 0.2 wt. % to about 5 wt. % palladium metal, based on a combined weight of the palladium metal and the alumina support.

A summary of the preferred catalyst and support, loading, and temperatures for use in the second reactor 240 of the two-reactor method as discussed above are summarized in Table 12 below.

TABLE 12
Summary of Catalyst (Pd, Pt, Rh) on Support (Carbon,
alpha alumina) - Two-Reactor Method Second Reactor -
Step (i) Hydrogenation Reaction

| | | Loading | Temperature |
|---|---|---|---|
| Catalyst | Support | (wt. %) | (° C.) |
| Pd | Activated carbon | 0.1-10 | 200-450 |
| Pd | Activated carbon | 0.1-10 | 220-400 |
| Pd | Activated carbon | 0.1-10 | 250-350 |
| Pd | Activated carbon | 0.1-10 | 275-350 |
| Pd | Activated carbon | 0.5-5 | 200-450 |
| Pd | Activated carbon | 0.5-5 | 220-400 |
| Pd | Activated carbon | 0.5-5 | 250-350 |
| Pd | Activated carbon | 0.5-5 | 275-350 |
| Pd | Activated carbon | 1-4 | 200-450 |
| Pd | Activated carbon | 1-4 | 220-400 |
| Pd | Activated carbon | 1-4 | 250-350 |
| Pd | Activated carbon | 1-4 | 275-350 |
| Pd | alpha(α)-Al₂O₃ | 0.1-10 | 150-350 |
| Pd | alpha(α)-Al₂O₃ | 0.1-10 | 150-250 |
| Pd | alpha(α)-Al₂O₃ | 0.2-5 | 150-350 |
| Pd | alpha(α)-Al₂O₃ | 0.2-5 | 150-250 |
| Pt | Activated carbon | 0.1-10 | 200-450 |
| Pt | Activated carbon | 0.1-10 | 220-400 |
| Pt | Activated carbon | 0.1-10 | 250-350 |
| Pt | Activated carbon | 0.1-10 | 275-350 |
| Pt | Activated carbon | 0.5-5 | 200-450 |
| Pt | Activated carbon | 0.5-5 | 220-400 |
| Pt | Activated carbon | 0.5-5 | 250-350 |
| Pt | Activated carbon | 0.5-5 | 275-350 |
| Pt | Activated carbon | 1-4 | 200-450 |
| Pt | Activated carbon | 1-4 | 220-400 |
| Pt | Activated carbon | 1-4 | 250-350 |
| Pt | Activated carbon | 1-4 | 275-350 |
| Pt | alpha(α)-Al₂O₃ | 0.1-10 | 150-350 |
| Pt | alpha(α)-Al₂O₃ | 0.1-10 | 150-250 |
| Pt | alpha(α)-Al₂O₃ | 0.2-5 | 150-350 |
| Pt | alpha(α)-Al₂O₃ | 0.2-5 | 150-250 |
| Rh | Activated carbon | 0.1-10 | 200-450 |
| Rh | Activated carbon | 0.1-10 | 220-400 |
| Rh | Activated carbon | 0.1-10 | 250-350 |
| Rh | Activated carbon | 0.1-10 | 275-350 |
| Rh | Activated carbon | 0.5-5 | 200-450 |
| Rh | Activated carbon | 0.5-5 | 220-400 |
| Rh | Activated carbon | 0.5-5 | 250-350 |
| Rh | Activated carbon | 0.5-5 | 275-350 |
| Rh | Activated carbon | 1-4 | 200-450 |
| Rh | Activated carbon | 1-4 | 220-400 |
| Rh | Activated carbon | 1-4 | 250-350 |
| Rh | Activated carbon | 1-4 | 275-350 |
| Rh | alpha(α)-Al₂O₃ | 0.1-10 | 150-350 |
| Rh | alpha(α)-Al₂O₃ | 0.1-10 | 150-250 |
| Rh | alpha(α)-Al₂O₃ | 0.2-5 | 150-350 |
| Rh | alpha(α)-Al₂O₃ | 0.2-5 | 150-250 |

j. Two-Reactor Method—Second Reactor Product Stream Composition

Referring again to FIG. 2, second product stream 242 exits from second reactor 240 of the two-reactor method and comprises desired product 1,1,2-trifluoroethane (HFC-143); desired intermediates 1-chloro-1,1,2-trifluoroethane (HCFC-133b), 1-chloro-1,2,2-trifluoroethane (HCFC-133), 1,2-dichloro-1,1,2-trifluoroethane (HCFC-123a); undesired byproducts HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), chloroethane (HCC-160), ethane (HC-170), 1,1-difluoroethane (HFC-152a), 1,1,1-trifluoroethane (HFC-143a), hydrogen chloride (HCl), hydrogen fluoride (HF); and unreacted hydrogen (H₂). Second product stream 242 may be conveyed to the distillation column 210 so that it may optionally be distilled and/or subjected to removal of acids to improve purity.

For a hydrogenation reaction of Step (i) carried out using each catalyst/support combination (each row) in Table 1 using a two-reactor method, referring to FIG. 2, the amount of 1,1,2-trifluoroethane (HFC-143) in the product composition of the second product stream 242 from the second reactor 240 of the two-reactor method may be at least 40 mol %, at least 50 mol %, or at least 60 mol %, for example, based on total moles of organic components of the composition.

For a hydrogenation reaction of Step (i) carried out using each catalyst/support combination (each row) in Table 1 using a two-reactor method, referring to FIG. 2, the amount of 1-chloro-1,1,2-trifluoroethane (HCFC-133b) in the product composition of the second product stream 242 from the second reactor 240 of the two-reactor method, if present, may be greater than 0.5 mol % and yet less than 40 mol %, less than 20 mol %, or less than 10 mol %, for example, based on total moles of organic components of the composition.

For a hydrogenation reaction of Step (i) carried out using each catalyst/support combination (each row) in Table 1 using a two-reactor method, referring to FIG. 2, the amount of 1-chloro-1,2,2-trifluoroethane (HCFC-133) in the product composition of the second product stream 242 from the second reactor 240 of the two-reactor method, if present, may be greater than 0.05% and yet less than 10 mol %, less than 5 mol %, or less than 1 mol %, for example, based on total moles of organic components of the composition.

For a hydrogenation reaction of Step (i) carried out using each catalyst/support combination (each row) in Table 1 using a two-reactor method, referring to FIG. 2, the amount of 1,2-dichloro-1,1,2-trifluoroethane (HCFC-123a) in the product composition of the second product stream 242 from the second reactor 240 of the two-reactor method, if present, may be less than 10 mol %, less than 5 mol %, or less than 0.1 mol %, and for each of the foregoing, greater than or equal to 0.01 mol %, for example, based on total moles of organic components of the composition.

For a hydrogenation reaction of Step (i) carried out using each catalyst/support combination (each row) in Table 1 using a two-reactor method, referring to FIG. 2, the total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) in the product composition of the second product stream 242 from the second reactor 240 of the two-reactor method may be at least 70 mol %, at least 80 mol %, or at least 90 mol %, and for each of the foregoing, less than or equal to 100 mol %, for example, based on total moles of organic components of the composition.

For example, for a hydrogenation reaction of Step (i) carried out using each catalyst/support combination (each row) in Table 1 using a two-reactor method, referring to FIG. 2, in the product composition of the second product stream 242 from the second reactor 240 of the two-reactor method, the amount of 1,1,2-trifluoroethane (HFC-143) may be at least 40 mol % and less than or equal to 99.45 mol %, the amount of 1-chloro-1,1,2-trifluoroethane (HCFC-133b) may be at least 0.5 mol % and less than or equal to 40 mol %, the amount of 1-chloro-1,2,2-trifluoroethane (HCFC-133) may be at least 0.05 mol % and less than or equal to 10 mol %, the amount of 1,2-dichloro-1,1,2-trifluoroethane (HCFC-123a) may be less than 10 mol % and greater than or equal to 0 mol %, based on the combined total moles of the HFC-143, HCFC-133b, HCFC-133, and HCFC-123a in the product composition.

The combined amount of HFC-143, HCFC-133b, HCFC-133, and HCFC-123a in the composition set forth in the preceding paragraph may be at least 70 mol % and less than or equal to 100% of the total moles of organic components of the product composition in the second product stream 242 from the second reactor 240 of the two-reactor method, while the combined amount of other components including undesired byproducts (e.g., HFC-143a, HC-170, HCC-160, HFC-152a, and HCFC-142 isomers) may be greater than or equal to 0 mol % and less than 30 mol % of the total moles of organic components of the product composition in the second product stream 242 from the second reactor 240 of the two-reactor method.

For example, for a hydrogenation reaction of Step (i) carried out using each catalyst/support combination (each row) in Table 1 using a two-reactor method, referring to FIG. 2, in the product composition of the second product stream 242 from the second reactor 240 of the two-reactor method, the amount of 1,1,2-trifluoroethane (HFC-143) may be at least 50 mol % and less than or equal to 99.45 mol %, the amount of 1-chloro-1,1,2-trifluoroethane (HCFC-133b) may be at least 0.5 mol % and less than or equal to 35 mol %, the amount of 1-chloro-1,2,2-trifluoroethane (HCFC-133) may be at least 0.05 mol % and less than or equal to 10 mol %, the amount of 1,2-dichloro-1,1,2-trifluoroethane (HCFC-123a) may be less than 5 mol % and greater than or equal to 0 mol %, based on the combined total moles of the HFC-143, HCFC-133b, HCFC-133, and HCFC-123a in the product composition.

The combined amount of HFC-143, HCFC-133b, HCFC-133, and HCFC-123a in the composition set forth in the preceding paragraph may be at least 80 mol % and less than or equal to 100% of the total moles of organic components of the product composition in the second product stream 242 from the second reactor 240 of the two-reactor method, while the combined amount of other components including undesired byproducts (e.g., HFC-143a, HC-170, HCC-160, HFC-152a, and HCFC-142 isomers) may be greater than or equal to 0 mol % and less than 20 mol % of the total moles of organic components of the product composition in the second product stream 242 from the second reactor 240 of the two-reactor method.

For example, for a hydrogenation reaction of Step (i) carried out using each catalyst/support combination (each row) in Table 1 using a two-reactor method, referring to FIG. 2, in the product composition of the second product stream 242 from the second reactor 240 of the two-reactor method, the amount of 1,1,2-trifluoroethane (HFC-143) may be at least 60 mol % and less than or equal to 100 mol %, the amount of 1-chloro-1,1,2-trifluoroethane (HCFC-133b) may be at least 0.5 mol % and yet less than or equal to 30 mol %, the amount of 1-chloro-1,2,2-trifluoroethane (HCFC-133) may be at least 0.05 mol % and less than or equal to 9 mol %, the amount of 1,2-dichloro-1,1,2-trifluoroethane (HCFC-123a) may be less than 1 mol % and greater than or equal to 0 mol %, based on the combined total moles of the HFC-143, HCFC-133b, HCFC-133, and HCFC-123a in the product composition.

The combined amount of HFC-143, HCFC-133b, HCFC-133, and HCFC-123a in the composition set forth in the preceding paragraph may be at least 90 mol % and less than or equal to 100% of the total moles of organic components of the product composition in the second product stream 242 from the second reactor 240 of the two-reactor method, while the combined amount of other components including undesired byproducts (e.g., HFC-143a, HC-170, HCC-160, HFC-152a, and HCFC-142 isomers) may be greater than or equal to 0 mol % and less than 10 mol % of the total moles of organic components of the product composition in the second product stream 242 from the second reactor 240 of the two-reactor method.

k. Two-Reactor Method—Amount of Increase in HFC-143 in the Product Stream Composition Provided by the Two-Reactor Method

Referring again to FIG. 2, the hydrogenation reaction in the second reactor 240 in the two-reactor method results in an increase in an amount of HFC-143 in the second product stream 242 exiting from the second reactor 240 compared to the bottom stream 238 from the fourth distillation column 234 before entering the second reactor 240. An amount of 1,1,2-trifluoroethane (HFC-143) in the second composition is increased by about 35 mol % to about 130 mol % relative to an amount of 1,1,2-trifluoroethane (HFC-143) in the first composition.

For a hydrogenation reaction of Step (i) carried out using each catalyst/support combination (each row) in Table 1 using a two-reactor method, referring to FIG. 2, an amount of HFC-143 in the second product stream 242 from the second reactor 240 is increased by at least 35 mol %, at least 40 mol %, or at least 45 mol %, or at least 50 mol %, or at least 60 mol %, or at least 70 mol %, or at least 80 mol %, or at least 90 mol %, or at least 100 mol %, or at least 110 mol %, or at least 120 mol %, and for each of the foregoing, less than or equal to 130 mol %, relative to an amount of HFC-143 in the bottom stream 238 from the fourth distillation column 234 before entering the second reactor 240.

In the present process according to the single-reactor method or the two-reactor method, undesired byproducts such as 1,1,1-trifluoroethane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), are not able to be converted to form 1,1,2-trifluoroethane (HFC-143), and therefore the present reaction methods and conditions may be tailored to avoid and/or minimize formation of such undesired byproducts.

### IV. Step (ii)

### General Process

The dehydrofluorination reaction of Step (ii) may be carried out in the vapor phase in a suitable reactor, for example a tubular reactor made from a material which is resistant to temperature and/or corrosion such as nickel and its alloys, including Hastelloy (for example, Hastelloy C276), Inconel (for example Inconel 600), Incoloy, and Monel wherein the vessels which may be lined with fluoropolymers.

The reactor may be first cleaned and flushed with an inert gas such as nitrogen, followed by packing with a catalyst such as those described below. The catalyst may be pretreated within the reactor such as by drying in the manner described further below, followed by metering the reactants into the rector to initiate the reaction.

The process flow may be in the down or up direction through a bed of the catalyst. Reactants may be flowed through a scrubber to remove undesired byproducts from the reaction, such as hydrogen fluoride (HF) and/or hydrogen chloride (HCl), and the reaction products may be collected by capture in a cooled cylinder, for example.

### Catalysts

The catalyst and process conditions play an important role in the dehydrofluorination reaction.

Suitable catalysts for the dehydrofluorination reaction include metal oxides such as chromium oxide (Cr₂O₃), aluminum oxide (Al₂O₃), iron oxide(Fe₂O₃), and magnesium oxide (MgO). Fluorination treatment of the catalyst may be conducted using anhydrous hydrogen fluoride (HF) under conditions effective to convert a portion of metal oxides into corresponding metal fluorides, such as via the procedure disclosed in U.S. Pat. No. 6,780,815 to Cerri et al., the disclosure of which is expressly incorporated by reference herein. Other suitable catalysts for the dehydrofluorination reaction include metal fluorides such as chromium fluoride (CrF₃), alumina fluoride (AlF₃), iron fluoride (FeF₃), magnesium fluoride (MgF₂), and various combinations of thereof.

Other metals, such as Pd, Pt, and Ni, may also be loaded onto the above fluorinated metal oxides, for example, via a wet impregnation process wherein a salt of the metal is exposed to the fluorinated metal oxide support in solution, followed by drying and then reduction with hydrogen gas. The metal catalysts supported on fluorinated metal oxide (resulting in a metal fluoride such as CrF₃, AlF₃, FeF₃, or MgF₂) supports are listed in Table 13 below.

TABLE 13
Supported Metal Catalysts - Step (ii) Dehydrofluorination Reaction

| Catalyst | Support |
|---|---|
| Pd | CrF₃ |
| Pd | AlF₃ |
| Pd | FeF₃ |
| Pd | MgF₂ |
| Pt | CrF₃ |
| Pt | AlF₃ |
| Pt | FeF₃ |
| Pt | MgF₂ |
| Ni | CrF₃ |
| Ni | AlF₃ |
| Ni | FeF₃ |
| Ni | MgF₂ |

Catalyst Loading (Pd, Pt, and Ni on CrF₃, AlF₃, FeF₃, or MgF₂)

The amount of metal loading on the support may be from about 0.01 wt. %, about 0.05 wt. %, about 0.1 wt. %, about 0.2 wt. %, about 0.3 wt. %, about 0.4 wt. %, about 0.5 wt. %, or about 1 wt. % to about 2 wt. %, about 3 wt. %, 5 wt. % 10 wt. %, or 20 wt. %, or 30 wt. %, or 40 wt. %, or 50 wt. % or within any range encompassed by two of the foregoing values as endpoints, based on a total weight of the catalyst and support, such as from about 0.01 wt. % to about 50 wt. %, from about 0.05 wt. % to about 40 wt. %, from about 0.1 wt. % to about 30 wt. %, from about 0.2 wt. % to about 20 wt. %, from about 0.3 wt. % to about 10 wt. %, from about 0.4 wt. % to about 5 wt. %, from about 0.5 wt. % to about 3 wt. %, or from about 1 wt. % to about 2 wt. %. For supported noble metal catalysts such as platinum or palladium, the metal loading may be ranged from about 0.01 wt. % to about 5 wt. %, preferably from about 0.05 wt. % to about 2 wt. %, and more preferably from about 0.1 wt. % to about 1 wt. %. Specific examples of additional suitable ranges are set forth below in Table 14.

TABLE 14
Loading for Catalysts (Pd, Pt, and Ni Loaded onto CrF₃,
AlF₃, FeF₃, or MgF₂) - Step (ii) Dehydrofluorination Reaction

| From (wt. %) | To (wt. %) |
|---|---|
| 0.01 | 10 |
| 0.01 | 5 |
| 0.01 | 3 |
| 0.01 | 2 |
| 0.01 | 1 |
| 0.05 | 10 |
| 0.05 | 5 |
| 0.05 | 3 |
| 0.05 | 2 |
| 0.05 | 1 |
| 0.1 | 10 |
| 0.1 | 5 |
| 0.1 | 3 |
| 0.1 | 2 |
| 0.1 | 1 |

When fluorinated alumina is used, the amount of metal loading on the support may be from about 0.01 wt. % to about 5 wt. %, preferably from about 0.05 wt. % to about 2 wt. %, most preferably from about 0.1 wt. % to about 1 wt. %.

### Catalyst BET Surface Area

The catalyst used in step (ii) may have a proper BET (Brunauer, Emmet, and Teller) surface area. The BET surface area of the catalyst may be as low as about 10 m²/g, about 20 m²/g, about 30 m²/g, about 40 m²/g, about 50 m²/g, about 60 m²/g, about 70 m²/g, about 80 m²/g, about 90 m²/g, about 100 m²/g, or as high as about 110 m²/g, about 120 m²/g, about 130 m²/g, about 140 m²/g, about 150 m²/g, about 175 m²/g, about 200 m²/g, about 225 m²/g, about 250 m²/g, about 300 m²/g, or within any range encompassed by any of the foregoing values as endpoints, such as from about 10 m²/g to about 300 m²/g, from about 20 m²/g to about 250 m²/g, from about 30 m²/g to about 225 m²/g, from about 40 m²/g to about 200 m²/g, from about 50 m²/g to about 175 m²/g, from about 60 m²/g to about 150 m²/g, from about 70 m²/g to about 140 m²/g, from about 80 m²/g to about 130 m²/g, from about 90 m²/g, to about 120 m²/g, or from about 100 m²/g to about 110 m²/g. For metal oxides catalysts, the BET surface area may be preferably greater than about 100 m²/g. For fluorinated metal oxides catalysts, the BET surface area may be preferably greater than about 20 m²/g. The BET analysis is the standard method for determining surface areas from nitrogen adsorption isotherms. The BET surface areas of catalysts may be measured using TriStar II Micromeritics instrument. Catalyst samples are degassed before the analysis using FlowPrep 060 instrument. Specific examples of additional suitable ranges are set forth below in Table 15.

TABLE 15
BET Surface Area of Catalysts (Cr₂O₃, Al₂O₃,
Fe₂O₃, MgO, CrF₃, AlF₃, FeF₃, MgF₂ or Pd, Pt, and
Ni Loaded onto CrF₃, AlF₃, FeF₃, or MgF₂) -
Step (ii) Dehydrofluorination Reaction

| From (m²/g) | To (m²/g) |
|---|---|
| 10 | 300 |
| 10 | 250 |
| 10 | 200 |
| 10 | 150 |
| 10 | 100 |
| 20 | 300 |
| 20 | 250 |
| 20 | 200 |
| 20 | 150 |
| 20 | 100 |
| 25 | 300 |
| 25 | 250 |
| 25 | 200 |
| 25 | 150 |
| 25 | 100 |

When fluorinated alumina is used, the BET surface area may be greater than about 10 m²/g, preferably greater than 20 m²/g, most preferably greater than 25 m²/g.

### Catalyst Pretreatment

The catalyst may be pretreated by drying at elevated temperatures, as low as about 200° C., about 250° C., about 300° C., about 350° C., about 360° C., about 370° C., or as high about 390° C., about 400° C., about 450° C., about 500° C., about 550° C., about 600° C., or within any range encompassed by two of the foregoing values as endpoints, such as from about 200° C. to about 600° C., from about 250° C. to about 550° C., from about 300° C. to about 500° C., from about 350° C. to about 450° C., from about 360° C. to about 400° C., or from about 370° C. to about 390° C. Specific examples of additional suitable ranges are set forth in Table 16 below.

TABLE 16
Pre-Treatment Drying Temperatures for Catalysts (Cr₂O₃, Al₂O₃, Fe₂O₃,
MgO, CrF₃, AlF₃, FeF₃, MgF₂ or Pd, Pt, and Ni Loaded onto CrF₃,
AlF₃, FeF₃, or MgF₂) - Step (ii) Dehydrofluorination Reaction

| From (° C.) | To (° C.) |
|---|---|
| 200 | 600 |
| 200 | 550 |
| 200 | 500 |
| 200 | 450 |
| 200 | 400 |
| 300 | 600 |
| 300 | 550 |
| 300 | 500 |
| 300 | 450 |
| 300 | 400 |
| 400 | 600 |
| 400 | 550 |
| 400 | 500 |
| 400 | 450 |

When fluorinated alumina is used, the catalyst may be pretreated by drying a temperature of from about 200° C. to about 600° C., preferably from about 300° C. to about 600° C., most preferably from about 400° C. to about 550° C.

As part of the catalyst activation, the catalyst may be exposed to an inert gas such as N₂. The pretreatment process may take as low as about 1 hour, about 2 hours, about 3 hours, about 4 hours, or as high about 5 hours, about 6 hours, about 10 hours, about 20 hours, or within any range encompassed by two of the foregoing values as endpoints such as from about 1 hour to about 20 hours, from about 2 hours to about 10 hours, from about 3 hours to about 6 hours, or from about 4 hours to about 5 hours.

When fluorinated alumina is used, the pretreatment process may take from about 1 hour to about 10 hours, preferably from about 2 hours to about 6 hours, most preferably from about 3 hours to about 5 hours.

### Reaction Conditions—Temperature

The temperature range for dehydrofluorination reaction may be as low as about 125° C., about 150° C., about 200° C., about 250° C., about 300° C., about 350° C., about 400° C., about 450° C., or as high as about 500° C., about 550° C., about 600° C., about 650° C., about 700° C., about 750° C., about 800° C. or within any range encompassed by two of the foregoing values as endpoints, such as from about 125° C. to about 800° C., from about 150° C. to about 750° C., from about 200° C. to about 650° C., from about 250° C. to about 600° C., from about 300° C. to about 550° C., about 350° C. to about 500° C., or about 400° C. to about 450° C. The temperature may be preferably from about 250° C. to about 450° C., and more preferably from about 300° C. to about 400° C. Specific examples of additional suitable ranges are set forth below in Table 17.

TABLE 17
Reaction Temperature when using Catalysts (Cr₂O₃, Al₂O₃, Fe₂O₃,
MgO, CrF₃, AlF₃, FeF₃, MgF₂ or Pd, Pt, and Ni Loaded onto CrF₃,
AlF₃, FeF₃, or MgF₂) - Step (ii) Dehydrofluorination Reaction

| From (° C.) | To (° C.) |
|---|---|
| 125 | 500 |
| 125 | 450 |
| 125 | 400 |
| 125 | 350 |
| 125 | 300 |
| 250 | 500 |
| 250 | 450 |
| 250 | 400 |
| 250 | 350 |
| 250 | 300 |
| 300 | 500 |
| 300 | 450 |
| 300 | 400 |
| 300 | 350 |

When fluorinated alumina is used, the reaction temperature may be from about 125° C. to about 500° C., preferably from about 250° C. to about 450° C., most preferably from about 300° C. to about 400° C.

### Reaction Conditions—Pressure

The pressure may be as little as about 1 psig, about 2 psig, about 3 psig, about 4 psig or about 5 psig, about 10 psig, about 15 psig, about 20 psig, about 25 psig, about 30 psig, about 35 psig, about 40 psig, about 50 psig, or within any range encompassed by two of the foregoing values as endpoints, such as from about 1 psig to about 50 psig, from about 2 psig to about 40 psig, from about 3 psig to about 35 psig, from about 4 psig to about 25 psig, from about 5 psig to about 20 psig, or from about 10 psig to about 15 psig. For example, the pressure may be from about 1 psig to about 50 psig, preferably from about 5 psig to about 30 psig, and more preferably from about 10 psig to about 20 psig. Specific examples of additional suitable ranges are set forth below in Table 18.

TABLE 18
Reaction Pressure when using Catalysts (Cr₂O₃, Al₂O₃, Fe₂O₃,
MgO, CrF₃, AlF₃, FeF₃, MgF₂ or Pd, Pt, and Ni Loaded onto
CrF₃, AlF₃, FeF₃, or MgF₂) - Step (ii) Dehydrofluorination Reaction

| From (psig) | To (psig) |
|---|---|
| 1 | 50 |
| 1 | 40 |
| 1 | 35 |
| 1 | 30 |
| 1 | 20 |
| 5 | 50 |
| 5 | 40 |
| 5 | 35 |
| 5 | 30 |
| 5 | 20 |
| 10 | 50 |
| 10 | 40 |
| 10 | 35 |
| 10 | 30 |
| 10 | 20 |

When fluorinated alumina is used, the reaction pressure may be from about 1 psig to about 50 psig, preferably from about 5 psig to about 30 psig, most preferably from about 10 psig to about 20 psig.

### Reaction Conditions—Contact Time

The contact time of the reactants with the catalyst may be as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints, such as from about 0.1 seconds to about 120 seconds, from about 1 second to about 60 seconds, from about 5 seconds to about 50 seconds, from about 10 seconds to about 40 seconds, from about 15 seconds to about 30 seconds, or from about 20 seconds to about 25 seconds. For example, the contact time may be from about 1 second to about 60 seconds. Specific examples of additional suitable ranges are set forth below in Table 19.

TABLE 19
Contact Time of Reactants with Catalysts (Cr₂O₃, Al₂O₃, Fe₂O₃,
MgO, CrF₃, AlF₃, FeF₃, MgF₂ or Pd, Pt, and Ni Loaded onto
CrF₃, AlF₃, FeF₃, or MgF₂) - Step (ii) Dehydrofluorination Reaction

| From (seconds) | To (seconds) |
|---|---|
| 1 | 120 |
| 1 | 60 |
| 1 | 40 |
| 1 | 30 |
| 1 | 20 |
| 5 | 120 |
| 5 | 60 |
| 5 | 40 |
| 5 | 30 |
| 5 | 20 |
| 10 | 120 |
| 10 | 60 |
| 10 | 40 |
| 10 | 30 |
| 10 | 20 |

When fluorinated alumina is used, the contact time may be from about 1 second to about 60 seconds, preferably from about 5 seconds to about 40 seconds, most preferably from about 10 seconds to about 30 seconds.

### Products—Cis/Trans Ratio

In the dehydrofluorination reactions of Step (ii), the cis/trans molar ratio of the 1,2-difluoroethylene in the product mixture may be as low as about 1, about 2, about 3, about 4, about 5, about 6, about 7, or as high as about 9, about 10, about 11, about 12, about 13, about 14, about 15 or within any range encompassed by two of the foregoing values as endpoints, such as from about 1 to about 15, from about 2 to about 14, from about 3 to about 13, from about 4 to about 12, from about 5 to about 11, from about 6 to about 10, or from about 7 to about 9. For example, the cis/trans ratio may be from about 2 to about 15.

When fluorinated alumina is used, the cis/trans molar ratio of the 1,2-difluoroethylene in the product mixture may be from about 1 to about 15, preferably from about 1 to about 10, most preferably from about 2 to about 7.

### Products—Selectivity

The selectivity to the desired 1,2-difluoroethylene product (the sum of 1232E and 1232Z) may be as low as about 80%, about 85%, about 89% about 90%, about 91%, about 92%, or as high as about 94%, about 95% about 96%, about 97%, about 98%, about 99%, or within any range encompassed by two of the foregoing values as endpoints, such as from about 80% to about 99%, from about 85% to about 98%, from about 89% to about 97%, from about 90% to about 96%, from about 91% to about 95%, or from about 92% to about 94%. Specific examples of additional suitable ranges are set forth below in Table 20.

TABLE 20
Selectivity to Desired Product using Catalysts (Cr₂O₃, Al₂O₃,
Fe₂O₃, MgO, CrF₃, AlF₃, FeF₃, MgF₂ or Pd, Pt, and Ni Loaded
onto CrF₃, AlF₃, FeF₃, or MgF₂) - Step (ii) Dehydrofluorination Reaction

| From (%) | To (%) |
|---|---|
| 85 | 99 |
| 85 | 98 |
| 85 | 97 |
| 85 | 96 |
| 85 | 95 |
| 90 | 99 |
| 90 | 98 |
| 90 | 97 |
| 90 | 96 |
| 90 | 95 |
| 95 | 99 |
| 95 | 98 |
| 95 | 97 |
| 95 | 96 |

When fluorinated alumina is used, the selectivity to the desired 1,2-difluoroethylene product (the sum of 1232E and 1232Z) may be from about 85% to about 99%, preferably from about 90% to about 99%, most preferably from about 95% to about 99%.

### Products—Conversion of the Starting Material

The conversion of the starting material to 1,2-difluoroethylene may be greater than about 10%, greater than about 20%, greater than about 30%, greater than about 40%, greater than about 50%, greater than about 60%, greater than about 70%, greater than about 80%, greater than about 90%, greater than about 95%, greater than about 99%, or within any range encompassed by two of the foregoing values as endpoints, such as from about 10% to about 99%, from about 20% to about 95%, from about 30% to about 80%, from about 40% to about 70%, or from about 50% to about 60%. Specific examples of additional suitable ranges are set forth below in Table 21.

TABLE 21
Conversion of Starting Material to 1.2-difluoroethylene
Catalysts (Cr₂O₃, Al₂O₃, Fe₂O₃, MgO, CrF₃, AlF₃, FeF₃, MgF₂
or Pd, Pt, and Ni Loaded onto CrF₃, AlF₃, FeF₃, or MgF₂) -
Step (ii) Dehydrofluorination Reaction

| From (%) | To (%) |
|---|---|
| 20 | 99 |
| 20 | 95 |
| 20 | 90 |
| 20 | 80 |
| 20 | 70 |
| 30 | 99 |
| 30 | 95 |
| 30 | 90 |
| 30 | 80 |
| 30 | 70 |
| 60 | 99 |
| 60 | 95 |
| 60 | 90 |
| 60 | 80 |
| 60 | 70 |

When fluorinated alumina is used, the conversion of the starting material to 1,2-difluoroethylene may be greater than about 20%, preferably greater than about 30%, most preferably greater than about 60%.

### Catalyst Regeneration

It may also be advantageous to periodically regenerate the catalyst after prolonged use while in place in the reactor. Regeneration of the catalyst may be accomplished by any means known in the art, for example, by passing air or air diluted with nitrogen over the catalyst at temperatures of from about 100° C. to about 400° C., preferably from about 200° C. to about 375° C., for from about 0.5 hour to about 3 days. This may be followed by hydrogen fluoride treatment at temperatures of from about 100° C. to about 400° C., preferably from about 200° C. to about 350° C., for fluorinated catalysts or hydrogen treatment at temperatures of from about 100° C. to about 400° C., preferably from about 200° C. to about 350° C. for supported transition metal catalysts.

When fluorinated alumina is used, the catalyst may be regenerated by passing air or air diluted with nitrogen over the catalyst at temperatures of from about 100° C. to about 400° C., preferably from about 200° C. to about 375° C., for from about 0.5 hour to about 3 days. This may be followed by hydrogen fluoride treatment at temperatures of from about 100° C. to about 400° C., preferably from about 200° C. to about 350° C. Additionally, the present process advantageously avoids and/or minimizes formation of 1,1,1,-trifluoroethane (HFC-143a) wherein the products of Step (ii), including trans-1,2-difluoroethylene (HFO-1132E), may include less than 5 mol %, less than 3 mol %, less than 1 mol %, less than 0.5 mol %, or less than 0.1 mol % of 1,1,1,-trifluoroethane (HFC-143a), based on total moles of the product composition.

Properties when the Catalyst is Fluorinated Alumina

When fluorinated alumina is used as a catalyst, the following properties may be present. The amount of metal loading on the support may be from about 0.01 wt. % to about 5 wt. %, preferably from about 0.05 wt. % to about 2 wt. %, most preferably from about 0.1 wt. % to about 1 wt. %. The BET surface area may be greater than about 10 m²/g, preferably greater than 20 m²/g, most preferably greater than 25 m²/g. The catalyst may be pretreated by drying a temperature of from about 200° C. to about 600° C., preferably from about 300° C. to about 600° C., most preferably from about 400° C. to about 550° C. The pretreatment process may take from about 1 hour to about 10 hours, preferably from about 2 hours to about 6 hours, most preferably from about 3 hours to about 5 hours. The reaction temperature may be from about 125° C. to about 500° C., preferably from about 250° C. to about 450° C., most preferably from about 300° C. to about 400° C. The reaction pressure may be from about 1 psig to about 50 psig, preferably from about 5 psig to about 30 psig, most preferably from about 10 psig to about 20 psig. The contact time may be from about 1 second to about 60 seconds, preferably from about 5 seconds to about 40 seconds, most preferably from about 10 seconds to about 30 seconds. The cis/trans molar ration of the 1,2-difluoroethylene in the product mixture may be from about 1 to about 15, preferably from about 1 to about 10, most preferably from about 2 to about 7. The selectivity to the desired 1,2-difluoroethylene product (the sum of 1232E and 1232Z) may be from about 85% to about 99%, preferably from about 90% to about 99%, most preferably from about 95% to about 99%. The conversion of the starting material to 1,2-difluoroethylene may be greater than about 20%, preferably greater than about 30%, most preferably greater than about 60%. The catalyst may be regenerated by passing air or air diluted with nitrogen over the catalyst at temperatures of from about 100° C. to about 400° C., preferably from about 200° C. to about 375° C., for from about 0.5 hour to about 3 days. This regeneration may be followed by hydrogen fluoride treatment at temperatures of from about 100° C. to about 400° C., preferably from about 200° C. to about 350° C.

### V. Step (iii)

### General Process

The 1,2-difluoroethylene (HFO-1132) obtained in Step (ii) above may be produced as a mixture containing both the trans-1,2-difluoroethylene (HFO-1132E) and cis-1,2-difluoroethylene (HFO-1132Z) isomers.

In step (iii), the cis-1,2-difluoroethylene (HFO-1132Z) isomer may be converted to the trans-1,2-difluoroethylene (HFO-1132E) isomer either by exposure to heat and/or a catalyst to yield a final product comprising at least 5 wt. % trans-1,2-difluoroethylene (HFO-1232E), at least 10 wt. % trans-1,2-difluoroethylene (HFO-1232E), at least 20 wt. % trans-1,2-difluoroethylene (HFO-1232E), at least 30 wt. % trans-1,2-difluoroethylene (HFO-1232E), or greater. The product stream from isomerization reactor can be further distilled to yield a final product comprising, consisting essentially of, or consisting of, the trans-1,2-difluoroethylene (HFO-1132E) isomer in high purity, such as at least about 95 wt. %, at least about 99.0 wt. %, at least about 99.9 wt. %, at least about 99.99 wt. % or greater.

The isomerization reaction may be conducted in any suitable reaction vessel or reactor, but it should preferably be constructed from materials which are resistant to corrosion such as nickel and its alloys, including Hastelloy (for example, Hastelloy C276), Inconel (for example Inconel 600), and Monel wherein the vessels which may be lined with fluoropolymers. These may be single pipe or multiple tubes packed with an isomerization catalyst

### Reaction Conditions—Temperature

The temperature range for the isomerization reaction may be as low as about 100° C., about 150° C., about 200° C., about 250° C., about 300° C., about 350° C., about 400° C., or as high as about 500° C., about 550° C., about 600° C., about 650° C., about 700° C., about 750° C., about 800° C. or within any range encompassed by two of the foregoing values as endpoints, such as from about 100° C. to about 800° C., from about 150° C. to about 750° C., from about 200° C. to about 700° C., from about 250° C. to about 650° C., from about 300° C. to about 600° C., from about 350° C. to about 550° C., or from about 400° C. to about 500° C. Specific examples of additional suitable ranges are set forth below in Table 22.

TABLE 22
Reaction Temperature - Step (iii) Isomerization Reaction

| From (° C.) | To (° C.) |
|---|---|
| 100 | 800 |
| 100 | 750 |
| 100 | 700 |
| 100 | 650 |
| 100 | 600 |
| 100 | 500 |
| 200 | 800 |
| 200 | 750 |
| 200 | 700 |
| 200 | 650 |
| 200 | 600 |
| 200 | 500 |
| 300 | 800 |
| 300 | 750 |
| 300 | 700 |
| 300 | 650 |
| 300 | 600 |
| 300 | 500 |

### Reaction Conditions—Pressure

The reaction may be conducted at atmospheric pressure, super-atmospheric pressure or under vacuum. The vacuum pressure can be from about 5 torr, about 10 torr, about 25 torr, about 50 torr, about 100 torr, about 150 torr, about 200 torr, about 250 torr, about 300 torr, to about 350 torr, 400 torr, about 450 torr, about 500 torr, about 550 torr, about 600 torr, about 650 torr, about 700 torr, about 760 torr, or any range using any of the foregoing values as endpoints, such as from about 5 torr to about 760 torr, about 10 torr to about 700 torr, about 25 torr to about 650 torr, about 50 torr to about 600 torr, about 100 torr to about 550 torr, about 150 torr to about 500 torr, about 200 torr about 450 torr, or about 250 torr to about 400 torr, or about 300 torr to about 350 torr. Specific examples of additional suitable ranges are set forth below in Table 23.

TABLE 23
Reaction Pressure - Step (iii) Isomerization Reaction

| From (torr) | To (torr) |
|---|---|
| 5 | 760 |
| 5 | 600 |
| 5 | 500 |
| 5 | 400 |
| 5 | 300 |
| 5 | 200 |
| 50 | 760 |
| 50 | 600 |
| 50 | 500 |
| 50 | 400 |
| 50 | 300 |
| 50 | 200 |
| 100 | 760 |
| 100 | 600 |
| 100 | 500 |
| 100 | 400 |
| 100 | 300 |
| 100 | 200 |

### Reaction Conditions—Contact Time

Contact time of the reactants with the catalyst may range from about 0.5 seconds, about 1 second, about 5 seconds, about 10 seconds, to about 20 seconds, about 30 seconds, about 60 seconds, or about 120 seconds, or any range using any of the foregoing values as endpoints, such as from about 0.5 seconds to about 120 seconds, about 1 second to about 60 seconds, about 5 seconds to about 30 seconds, or about 10 seconds to about 20 seconds. However, longer or shorter times can be used.

### Reaction Conditions—Environment

The reaction may also be conducted in an inert atmosphere substantially in the absence of oxygen. For example, the amount of oxygen present during the reaction may be less than 15 wt. %, less than 10 wt. %, less than 5 wt. %, or less than 1 wt. %, or any range using any two of the foregoing values as endpoints, such as from about 1 wt. % to about 15 wt. %, or about 5 wt. % to about 10 wt. %, based on a total weight of the reactants in the reactor.

The reaction may also be conducted substantially in the absence of water. For example, the amount of water present during the reaction may be less than 5 wt. %, less than 1 wt. %, less than 0.5 wt. %, or less than 0.05 wt. %, or any range using any two of the foregoing values as endpoints, such as 0.05 wt. % to about 5 wt. %, or about 0.5 wt. % to about 1 wt. %, based on a total weight of the reactants in the reactor.

### EXAMPLES

### Example 1

### Reaction of Intermediates to Produce HFC-143 in a Single-Reactor Method

Example 1 shows conversion of a feed containing 17±2% HCFC-123a and 83±2% CFC-113 using Pd/C catalysts. The purpose of these experiments was to evaluate the recyclability of reaction intermediates in the process of CFC-113 conversion to HFC-143. A condition can be considered suitable for recycling of the HCFC-123a molecule if its content in the product stream is less than that in the feed.

The experimental apparatus used includes a feed system containing gas flow controllers for N₂ and H₂ and a Micromotion mass flow meter connected to a research control valve (RCV) controlling the organic flow rate. The reactor consists of a one inch 316 SS tube packed with the catalyst. A thermocouple is inserted into the middle of the catalyst bed to read the operating temperature. The pressure control system consists of a RCV which is controlling the pressure by getting feedback from the pressure transducer placed after the reactor. For GC analysis, samples are taken after the reactor using a sample bag filled with ˜50 g of water to capture hydrogen chloride (HCl) and hydrogen fluoride (HF). Before the GC analysis, the sample bag is heated at 60° C. for one hour to assure that all the organic content is in the gas phase. Then, a sample is taken using a syringe and injected into the GC-FID instrument for analysis.

Table 24 shows the GC-FID area percentages of the product stream for three different catalysts at specified temperatures. The organic feed rate was 10 g/h, H₂ feeds rate was 150 ml/min, catalyst volume was 50 ml, and pressure was 45 psig.

At temperatures >225° C. as shown in Table 24, all the catalysts showed near complete conversion of CFC-113, and a HCFC-123a concentration lower than that in the feed indicating HCFC-123a consumption rate is more than its generation rate. With increasing reaction temperature, the amount of unconverted HCFC-123a was decreased. Catalyst with higher Pd loading requires lower temperature to achieve near complete conversion (>99%) of HCFC-123a, based on GC-FID area percentages. For example, near complete conversion of HCFC-123a was observed at 250° C. over the 4% Pd/C catalyst, while it was noted at about 280° C. over the 2% Pd/C one.

The BET (Brunauer, Emmet, and Teller) analysis is the standard method for determining surface areas from nitrogen adsorption isotherms. The BET surface areas of catalysts may be measured using TriStar II Micromeritics instrument. Catalyst samples are degassed at 150° C. using FlowPrep 060 instrument before BET analysis. The BET surface area of 4% Pd/C catalyst in Example 1 was 1661.6 m²/g. The BET surface area of 2% Pd/C catalyst in Example 1 was 207.7 m²/g. The BET surface area of 1% Pd/C catalyst in Example 1 was 1042.2 m²/g.

TABLE 24
Product compositions after the reactor for a feed containing 17 ±
2% HCFC-123a and 83 ± 2% CFC-113 using Pd/C catalysts

| | | GC-FID percent area | | | | | |
|---|---|---|---|---|---|---|---|
| | Temp | | | | | | total |
| Catalyst | (° C.) | R-143 | R-133b | R-133 | R-123a | R-113 | desired |
| 4% | 230 | 66.35 | 13.43 | 6.34 | 5.19 | 0.00 | 91.31 |
| Pd/C | 250 | 71.36 | 12.99 | 5.09 | 0.15 | 0.00 | 89.59 |
| | 281 | 78.44 | 5.93 | 1.47 | 0.03 | 0.01 | 85.88 |
| 2% | 228 | 44.98 | 29.50 | 3.12 | 12.55 | 0.00 | 90.15 |
| Pd/C | 250 | 60.25 | 22.83 | 3.93 | 2.55 | 0.00 | 89.56 |
| | 281 | 71.93 | 12.72 | 1.48 | 0.09 | 0.00 | 86.21 |
| 1% | 251 | 56.64 | 10.74 | 2.46 | 13.31 | 0.01 | 83.16 |
| Pd/C | 281 | 63.65 | 12.70 | 3.37 | 1.54 | 0.04 | 81.30 |

### Example 1a

This example shows conversion of pure CFC-113 and pure HCFC-123a (purity >99%) to HFC-143 using a 1% Pd/C catalyst at 200° C. The reaction setup was same as Example 1. Table 25 shows the GC-FID area percentages of the product stream. The organic feed rate was 10 g/h, H₂ feeds rate was 150 ml/min, catalyst volume was 50 ml, and pressure was 45 psig. The CFC-113 conversion under these conditions was more than 99.9%, based on total moles of organic components of the composition. Large quantities of HCFC-123a, HCFC-133b, and HCFC-133 intermediates were generated, but these intermediates may be recycled. The conversion of pure HCFC-123a under these conditions was 8.74% based on total moles of organic components of the composition, indicating that HCFC-123a is a lot less reactive compared to CFC-113. The total amount of undesired byproducts, including the amount of major byproduct R-152a, was less than 25%, or less than 20%, based on total moles of organic components of the composition.

For the data in Table 25, the temperature was 200±1° C., pressure was 45 psig, organic feed rate was 10 g/h, H₂ feeds rate was 150 ml/min, and catalyst volume was 50 ml. The BET surface area of 1% Pd/C catalyst in Example 1a was 865.4 m²/g.

TABLE 25
Conversion of 99.9% pure CFC-113 feed using 1% Pd/C

| | | Selectivity (%) | | | | | | | |
|---|---|---|---|---|---|---|---|---|---|
| | Conv. | R- | R- | R- | R- | R- | R- | R- | Total |
| Feed | (%) | 143 | 1123 | 133b | 133 | 123a | 113 | 152a | undesired |
| R-113 | 99.94 | 26.84 | 13.74 | 6.24 | 2.32 | 30.26 | N/A | 12.25 | 19.04 |
| R-123a | 8.74 | 50.23 | 0.00 | 8.58 | 10.76 | N/A | 0.00 | 19.91 | 24.54 |

### Example 1b

This example shows conversion of a feed containing 17±2% HCFC-123a and 83±2% CFC-113 using Pd/C and Pd/Al₂O₃ catalysts at temperatures ≤200° C. The reaction set up was similar to Example 1. The purpose of these experiments was to evaluate the recyclability of reaction intermediates in the process of CFC-113 conversion to HFC-143. A condition can be considered suitable for recycling of the HCFC-123a molecule if its content in the product stream is less than that in the feed.

Table 26 shows the GC-FID analysis results (area percentages) for the product stream. The organic feed rate was 10 g/h, H₂ feeds rate was 150 ml/min, and the reactor pressure was 45 psig. For the 1% Pd/C catalyst, 50 ml of the catalyst was used without dilution. For the 0.3% Pd/theta(θ)-Al₂O₃ catalyst, 10 ml of the catalyst was diluted with 40 ml of 316 SS mesh packing materials (1.8 inch). For both catalysts, the CFC-123a content was higher than the feed composition at temperatures ≤200° C. These results show HCFC-123a generation rate is more than its consumption rate at temperatures ≤200° C. and, therefore, temperatures >200° C. may be preferable for recycling HCFC-123a and its conversion to HFC-143.

For the data in Table 26, the organic feed rate was 10 g/h, H₂ feeds rate was 150 ml/min, catalyst volume was 50 ml, and pressure was 45 psig. The BET surface area of 1% Pd/C catalyst in Example 1 b was 865.4 m²/g. The BET surface area of 0.3% Pd/theta(θ)-Al₂O₃ catalyst in Example 1 b was 38.1 m²/g.

TABLE 26
Conversion of a feed containing 17 ± 2% HCFC-123a
and 83 ± 2% CFC-113 using Pd catalysts

| | | GC-FID percent area | | | | |
|---|---|---|---|---|---|---|
| | Temp | R- | R- | R- | R- | R- |
| Catalyst | (° C.) | 143 | 133b | 133 | 123a | 113 |
| 0.3% Pd/theta(θ)-Al₂O₃ | 164 | 12.79 | 16.88 | 0.22 | 21.69 | 45.08 |
| | 200 | 31.00 | 24.03 | 0.44 | 26.01 | 0.19 |
| 1% Pd/C | 165 | 27.16 | 24.92 | 0.56 | 35.91 | 3.72 |
| | 200 | 41.03 | 16.08 | 1.36 | 29.53 | 0.03 |

### Example 2

### Second Reaction in a Two-Reactor Method Using Intermediates to Produce HFC-143

This example shows conversion of (HCFC-133b and HCFC-133)/HCFC-123a which are intermediates in the process of CFC-113 conversion to HFC-143 in a two-reactor method.

The organic feed was obtained after distillation of reaction products formed and collected in a first reactor of the two-reactor method, which was operated mostly at 200° C. or lower. In summary, ˜500 gram of the distillation column overhead was collected in a 1 L SS cylinder. Note that distillation column overhead initially contained 0.23 wt % hydrogen fluoride (HF) and 0.03 wt % hydrogen chloride (HCl) (see below for the analysis procedure); using this feed led to catalyst deactivation. Therefore, we needed to remove acids.

The collected distillation column overhead was scrubbed by passing it through a ½ inch PFA tubbing packed with 14 inches of its length with CLR-204 to remove HCl, 9 inches of P-188 to remove hydrogen fluoride (HF), 12 inches of activated 3 A molecular sieves to remove water, and 3 inches of Drierite as water indicator. The material at the end of the acid-removal tube was collected in another clean 1 L SS cylinder placed in a dry-ice bath. The collected product did not show any hydrogen chloride (HCl) and hydrogen fluoride (HF), showing the effectiveness of the acid-removal procedure.

The materials collected in product collection cylinders (PCC) were generally analyzed using the following procedure. A known amount of the sample (1-3 grams) was taken using a Tedlar sample bag containing known amount of water (˜50 grams). After heating the sample bag at 60° C., 0.25 ml of the gas sample was taken using a GC-syringe (also heated in the oven at 60° C.) and immediately injected into a GC instrument. The acid content in the gas was determined using ion chromatography (IC) analysis of the water inside the Tedlar bag.

The experimental setup for a two-reactor method was similar to Example 1 and as shown in FIG. 2. 50 ml of the 4% Pd/C catalyst was charged to the second reactor 240 in the two-reactor method. Reaction was performed at 45 psig, with an organic feed rate of 10 g/h and H₂ feeds rate of 150 ml/min. A sample was taken right before the stream reaches the second reactor 240 in the two-reactor method, showing the following feed composition (based on GC-FID area percentages): 54.56% 1,1,2-trifluoroethane (HFC-143), 30.58% 1-chloro-1,1,2-trifluoroethane (HCFC-133b), 0.78% 1-chloro-1,2,2-trifluoroethane (HCFC-133), 2.43% 1,2-dichloro-1,1,2-trifluoroethane (HCFC-123a), and 3.60% 1,1,2-trichloro-1,2,2-trifluoroethane (CFC-113).

Table 27 shows the GC-FID area percentages of the product stream (i.e., stream 242 after the second reactor 240 of the two-reactor method). At 250° C., there was near complete conversion of 1,1,2-trichloro-1,2,2-trifluoroethane (CFC-113) and 1,2-dichloro-1,1,2-trifluoroethane (HCFC-123a) from the feed, 22% conversion of 1-chloro-1,1,2-trifluoroethane (HCFC-133b), and small increase in the 1-chloro-1,2,2-trifluoroethane (HCFC-133) content, based on total moles of organic components of the composition. At 280° C., there was near complete conversion of 1,1,2-trichloro-1,2,2-trifluoroethane (CFC-113) and 1,2-dichloro-1,1,2-trifluoroethane (HCFC-123a) from the feed, 52% conversion of 1-chloro-1,1,2-trifluoroethane (HCFC-133b), and 72% conversion of 1-chloro-1,2,2-trifluoroethane (HCFC-133), based on total moles of organic components of the composition.

For the data in Table 27, The organic feed rate was 10 g/h, H₂ feeds rate was 150 ml/min, catalyst volume was 50 ml, and pressure was 45 psig.

TABLE 27
Conversion of a feed containing 54.56% HFC-143,
30.58% HCFC-133b, 0.78% HCFC-133, 2.43% HCFC-123a,
and 3.60% CFC-113 using 4% Pd/C catalysts

| | GC-FID percent area | | | | |
|---|---|---|---|---|---|
| Temp/° C. | R-143 | R-133b | R-133 | R-123a | R-113 |
| 251 | 65.54 | 23.81 | 0.87 | 0.04 | 0.00 |
| 279 | 68.02 | 14.63 | 0.22 | 0.01 | 0.00 |

### Example 3

### Single-Reactor Method Using Pure CFC-113 to Produce HFC-143

This example shows conversion of pure CFC-113 to HFC-143 using 4% Pd/C catalyst at temperatures ≥250° C. (in a single-reactor method). The experimental setup was similar to Example 1. The organic feed rate was 10 g/h, H₂ feeds rate was 150 ml/min, catalyst volume was 50 ml, and pressure was 45 psig.

Table 28 shows the GC-FID area percentages of the product stream after the reactor (e.g., stream 108 after reactor 106 as shown in FIG. 1). CFC-113 conversion was 100% at all temperatures, based on total moles of organic components of the composition. The total of recyclable reaction intermediates (HCFC-133b and HCFC-133)/HCFC-123a formed as a function of temperature were 23.93% at 251° C., 22.78% at 256° C., and 20.21% at 260° C., based on total moles of organic components of the composition. Less of reaction intermediates are generated at higher temperatures. It appears that the reaction intermediates can be further reacted at temperatures >250° C.

TABLE 28
Conversion of pure CFC-113 using 4% Pd/C catalysts
as a function of reactor temperature

| | GC-FID percent area | | | | | |
|---|---|---|---|---|---|---|
| | R- | R- | R- | R- | R- | Total |
| Temp / C. | 143 | 133b | 133 | 123a | 113 | desired |
| 251 | 70.89 | 18.65 | 2.04 | 3.24 | 0.00 | 94.81 |
| 256 | 71.64 | 18.62 | 2.02 | 2.14 | 0.00 | 94.42 |
| 260 | 73.46 | 17.41 | 1.91 | 1.20 | 0.00 | 93.99 |

### Example 3a

This example shows conversion of pure CFC-113 to HFC-143 using 4% Pd/C catalyst at 200° C. (in a single-reactor method). The experimental setup was similar to Example 1. The organic feed rate was 10 g/h, H₂ feeds rate was 150 ml/min, catalyst volume was 50 ml, and pressure was 45 psig.

Table 29 shows the GC-FID area percentages of the product stream after the reactor (e.g., in stream 108 after the reactor 106 as shown in FIG. 1). CFC-113 conversion was 100%, based on total moles of organic components of the composition. The total of recyclable reaction intermediates (HCFC-133b and HCFC-133)/HCFC-123a formed as a function of temperature was 38.29% at 200° C., based on total moles of organic components of the composition. More of reaction intermediates are generated at 200° C.

TABLE 29
Conversion of pure 1,1,2-trichloro-1,2,2-trifluoroethane
(CFC-113) using 4% Pd/C catalysts at 200° C.

| | GC-FID percent area | | | | | |
|---|---|---|---|---|---|---|
| | R- | R- | R- | R- | R- | Total |
| Temp / ° C. | 143 | 133b | 133 | 123a | 113 | desired |
| 200 | 55.37 | 18.16 | 2.51 | 17.62 | 0.00 | 93.66 |

### Example 4

### Single-Reactor Method to Produce HFC-143

This example shows conversion of CFC-113 to HFC-143 in a single-reactor and using the same reactor for the conversion of recycled reaction intermediates HCFC-123a, HCFC-133b, and HCFC-133 to HFC-143.

The overall layout is as described above in connection with FIG. 1, and the feed system and the reactor are as set forth above in Example 1.

The reactor includes a one inch 316 SS tube packed with 50 ml of 4% Pd/C catalyst. A thermocouple is inserted into the middle of the catalyst bed to read the operating temperature. The pressure control system consists of a RCV which controls the pressure by getting feedback from the pressure transducer placed after the reactor. The reactor is operated at 250° C., with 10 g/h organic feed rate, and 150 ml/min H₂ feed rate.

The product composition in the stream 108 after the reaction in reactor 106 is determined using GC-FID analysis of the samples taken from the product stream 108 after the reaction in reactor 106 using a sample bag filled with ˜50 g of water to capture hydrogen chloride (HCl) and hydrogen fluoride (HF). Before the GC analysis, the sample bag is heated at 60° C. for one hour to assure that all the organic content is in the gas phase. The product stream 108 from the reactor 106 in the first run comprises the mixture provided in the table below.

TABLE 30
Composition of product stream 108 from the reactor 106

| mole % (desired) | | | | | | mole % (undesired) | | | | | | |
|---|---|---|---|---|---|---|---|---|---|---|---|---|
| R- | R- | R- | R- | R- | | R- | R- | R- | R- | R-142 | | |
| 143 | 133b | 133 | 123a | 113 | total | 143a | 170 | 152a | 160 | isomers | Other* | total |
| 70.89 | 18.65 | 2.04 | 3.24 | 0.00 | 94.82 | 0.11 | 0.92 | 1.95 | 0.41 | 1.15 | 0.65 | 5.19 |

Note that the provided percentages are mole percentages regarding only the organic content of the product stream 108 from the reactor 106. The product stream 108 from the reactor 106 also contains unreacted H₂, hydrogen chloride (HCl) generated from dehydrochlorination reactions, and hydrogen fluoride (HF) generated from dehydrofluorination side-reactions.

The product stream 108 from the reactor 106 is sent through a first distillation column 110 to recover the unreacted hydrogen (H₂) from the top stream. The bottom stream 116 from the first distillation column 110 is sent to a second distillation column 118 to remove hydrogen chloride (HCl) and ethane (HC-170) in the top stream 120. The bottom stream 122 from the second distillation column 118 is sent to a scrubber 124 to remove hydrogen fluoride (HF) and then to a third distillation column 128 to remove the low boiling point molecules such as HFC-152a, HCFC-142b, HFO-1123, and HFC-143a through the top stream 130 from the third distillation column 128.

The bottom stream 132 from the third distillation column 128 is sent to a fourth distillation column 134. The top stream 136 from the fourth distillation column 134 comprises HFC-143 with a purity greater than 91%, with less than 9% of impurities such as HCFC-133b and HCFC-133. The bottom stream 138 from the fourth distillation column 134 comprises the following mixture provided in the table below.

TABLE 31
Composition of Bottom Stream

| mole % (desired) | | | | | | mole % (undesired) | | | | | | |
|---|---|---|---|---|---|---|---|---|---|---|---|---|
| R- | R- | R- | R- | R- | | R- | R- | R- | R- | R-142 | | |
| 143 | 133b | 133 | 123a | 113 | total | 143a | 170 | 152a | 160 | isomers | Other | total |
| 45.85 | 37.18 | 5.37 | 8.53 | 0.00 | 96.94 | 0.00 | 0.00 | 0.00 | 0.02 | 3.03 | 0.01 | 3.06 |

The bottom stream 138 from the fourth distillation column 134 is sent back to the reactor 106 via a recycle stream to recycle the HCFC-133b, HCFC-133, and HCFC-123a intermediates.

### Example 5

### Two-Reactor Method to Produce HFC-143

This example shows conversion of CFC-113 to HFC-143 in a first reactor and conversion of recycled reaction intermediates HCFC-123a, HCFC-133b, and HCFC-133 to HFC-143 in a second reactor.

The overall layout is as described above in connection with FIG. 2, and the feed system and the reactor are as set forth above in Example 1.

The reactor includes a one-inch 316 SS tube packed with 50 ml of 4% Pd/C catalyst. A thermocouple is inserted into the middle of the catalyst bed to read the operating temperature. The pressure control system consists of a RCV which controls the pressure by getting feedback from the pressure transducer placed after the reactor. The reactor is operated at 250° C., with 10 g/h organic feed rate, and 150 ml/min H₂ feed rate.

The product composition in the product stream 208 from the first reactor 206 is determined using GC-FID analysis of the samples taken after the hydrogenation reaction using a sample bag filled with ˜50 g of water to capture hydrogen chloride (HCl) and hydrogen fluoride (HF). Before the GC analysis, the sample bag is heated at 60° C. for one hour to assure that all the organic content is in the gas phase. The product stream 208 from the first reactor 206 comprises the mixture provided in the table below.

TABLE 32
Composition of product stream 208 from first reactor 206

| mole % (desired) | | | | | | mole % (undesired) | | | | | | |
|---|---|---|---|---|---|---|---|---|---|---|---|---|
| R- | R- | R- | R- | R- | | R- | R- | R- | R- | R-142 | | |
| 143 | 133b | 133 | 123a | 113 | total | 143a | 170 | 152a | 160 | isomers | Other* | total |
| 70.89 | 18.65 | 2.04 | 3.24 | 0.00 | 94.82 | 0.10 | 0.92 | 1.95 | 0.41 | 1.15 | 0.65 | 5.18 |

Note that the provided percentages are mole percentages regarding only the organic content of the product stream. The product stream also contains unreacted H₂, hydrogen chloride (HCl) generated from dehydrochlorination reactions, and hydrogen fluoride (HF) generated from dehydrofluorination side-reactions.

The product stream 208 from the first reactor 206 is sent through a first distillation column 210 to recover the unreacted H₂. The bottom stream 216 from the first distillation column 210 is sent to a second distillation column 218 to remove hydrogen chloride (HCl) and ethane (HC-170). The bottom stream 222 from the second distillation column 218 is sent to a scrubber 224 to remove hydrogen fluoride (HF) and then to the third distillation column 228 to remove the low boiling point molecules such as 1,1-difluoroethane (HFC-152a), HCFC-142b isomers (1-chloro-1,1-difluoroethane), 1,1,2-trifluoroethene (HFO-1123), and 1,1,1-trifluoroethane (HFC-143a) via the top stream 230 from the third distillation column 228. The bottom stream 232 from the third distillation column 228 is sent to the fourth distillation column 234. The top stream 236 from the fourth distillation column 234 comprises HFC-143 with a purity greater than 91%, with less than 9% of impurities such as HCFC-133b and HCFC-133. The bottom stream 238 from the fourth distillation column 234 (in the first run before recycling intermediates) comprises the following mixture provided in the table below.

TABLE 33
Composition of Bottom Stream 238 from Fourth Distillation Column 234

| mole % (desired) | | | | | | mole % (undesired) | | | | | | |
|---|---|---|---|---|---|---|---|---|---|---|---|---|
| R- | R- | R- | R- | R- | | R- | R- | R- | R | R-142 | | |
| 143 | 133b | 133 | 123a | 113 | total | 143a | 170 | 152a | 160 | isomers | Other* | total |
| 45.85 | 37.18 | 5.37 | 8.53 | 0.00 | 96.94 | 0.00 | 0.00 | 0.00 | 0.02 | 3.03 | 0.01 | 3.06 |

The bottom stream 238 from the fourth distillation column 234 is also sent to the second reactor 240 of the two-reactor method. The second reactor 240 consists of a one-inch 316 SS tube packed with 50 ml of 4% Pd/C catalyst. A thermocouple is inserted into the middle of the catalyst bed to read the operating temperature. The pressure control system consists of a RCV which controls the pressure by getting feedback from the pressure transducer placed after the second reactor 240. The second reactor 240 is operated at 45 psig, 280° C., with 10 g/h organic feed rate, and 150 ml/min H₂ feed rate. The product composition in second product stream 242 from the second reactor 240 is determined using GC-FID analysis of the samples taken after the second reactor 240 using a sample bag filled with ˜50 g of water to capture hydrogen chloride (HCl) and hydrogen fluoride (HF). Before the GC analysis, the sample bag is heated at 60° C. for one hour to assure that all the organic content is in the gas phase. Under these conditions, the conversion of HCFC-133b, HCFC-133, and HCFC-123a are 52%, 72%, and 100%, respectively, based on total moles of organic components of the composition. The chloroethane (HCC-160) and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) molecules are also converted to ethane (HC-170) and 1,2-difluoroethane (HFC-152) in the reactor (˜90% conversion based on total moles of organic components of the composition).

The product stream 242 from the second reactor 240 (in the first run of recycling intermediates) comprises the following mixture provided in the table below.

TABLE 34
Composition of product stream from second reactor

| mole % (desired) | | | | | | mole % (undesired) | | | | | | |
|---|---|---|---|---|---|---|---|---|---|---|---|---|
| R- | R- | R- | R- | R- | | R- | R- | R- | R- | R-142 | | |
| 143 | 133b | 133 | 123a | 113 | total | 143a | 170 | 152a | 160 | isomers | Other* | total |
| 73.39 | 17.85 | 1.50 | 0.00 | 0.00 | 92.75 | 0.18 | 1.41 | 3.64 | 0.10 | 0.72 | 1.20 | 7.25 |

This product stream 242 from the second reactor 240 is mixed with the product stream 208 from the first reactor 206 and sent to the same distillation columns and scrubber as described above.

### Example 6

### Single-Reactor Method Using Pt/C as Catalyst

Example 6 shows conversion of feed containing 83% CFC-113 and 17% HCFC-123a using 0.5% Pt/C catalyst and the same overall layout and reaction conditions as Example 1. 50 ml of neat catalyst was loaded into the reactor. The organic flow rate was 10 g/h and hydrogen flow rate was 300 ml/min. Reaction was performed at 45 psig. The GC-FID analysis results of the reactor effluent as a function of catalyst bed temperature are shown in tables below.

Tables 35 and 36 show the GC-FID area percentages of the product stream for the Pt/C catalyst at specified temperatures.

TABLE 35
Desired intermediates in product compositions after
the reactor for a feed containing 83% CFC-113
and 17% HCFC-123a using 0.5% Pt/C catalysts

GC-FID percent area

| Temp | R- | R- | R- | R- | R- | total |
|---|---|---|---|---|---|---|
| (° C.) | 143 | 133b | 133 | 123a | 113 | desired |
| 219 | 11.28 | 12.23 | 0.59 | 42.15 | 1.49 | 67.73 |
| 229 | 11.97 | 14.37 | 0.69 | 34.67 | 0.02 | 61.71 |
| 251 | 14.75 | 15.58 | 1.00 | 28.00 | 0.02 | 59.34 |
| 282 | 21.10 | 14.12 | 1.59 | 16.85 | 0.01 | 53.66 |

TABLE 36
Undesired byproducts in product compositions after
the reactor for a feed containing 83% CFC-113
and 17% HCFC-123a using 0.5% Pt/C catalysts

| | GC-FID percent area | | | | | | | |
|---|---|---|---|---|---|---|---|---|
| Temp | R- | R- | R- | R-142 | R- | R- | | total |
| (° C.) | 152a | 170 | 160 | isomers | 1132a | 150 | Other | undesired |
| 219 | 3.55 | 6.60 | 3.50 | 0.71 | 0.03 | 0.13 | 17.77 | 32.27 |
| 229 | 4.24 | 7.87 | 5.80 | 0.82 | 0.11 | 0.40 | 19.07 | 38.29 |
| 251 | 5.15 | 9.26 | 8.70 | 0.95 | 0.31 | 0.95 | 15.35 | 40.66 |
| 282 | 6.39 | 15.83 | 9.95 | 1.29 | 0.58 | 1.22 | 11.10 | 46.34 |

### Example 7

### Single-Reactor Method Using Rh/Al₂O₃ as Catalyst

Example 7 shows conversion of feed containing 83% CFC-113 and 17% HCFC-123a using 0.5% Rh/Al₂O₃ catalyst and the same overall layout and reaction conditions as Example 1. 10 ml of the catalyst was diluted with 40 ml of SS mesh. The organic flow rate was 10 g/h and hydrogen flow rate was 300 ml/min. Reaction was performed at 45 psig. The GC-FID analysis results of the reactor effluent as a function of catalyst bed temperature are shown in tables below.

Tables 37 and 38 show the GC-FID area percentages of the product stream for the Rh/Al₂O₃ catalyst at specified temperatures.

TABLE 37
Desired intermediates in product compositions after
the reactor for a feed containing 83% CFC-113 and 17% HCFC-123a
using 0.5% Rh/Al₂O₃ catalysts

GC-FID percent area

| Temp | R- | R- | R- | R- | R- | total |
|---|---|---|---|---|---|---|
| (° C.) | 143 | 133b | 133 | 123a | 113 | desired |
| 161 | 4.81 | 15.07 | 0.56 | 19.09 | 53.92 | 93.44 |
| 197 | 9.47 | 10.55 | 1.53 | 27.13 | 42.67 | 91.35 |

TABLE 38
Undesired byproducts in product compositions after
the reactor for a feed containing 83% CFC-113 and 17% HCFC-123a
using 0.5% Rh/Al₂O₃ catalysts

| | GC-FID percent area | | | | | | | |
|---|---|---|---|---|---|---|---|---|
| Temp | R- | R- | R- | R-142 | R- | R- | | total |
| (° C.) | 152a | 170 | 160 | isomers | 1132a | 150 | Other | undesired |
| 161 | 0.92 | 2.83 | 0.79 | 0.29 | 0.00 | 0.12 | 1.62 | 6.56 |
| 197 | 1.12 | 3.50 | 0.80 | 0.33 | 0.00 | 0.13 | 2.77 | 8.65 |

### Example 8

### Two-Reactor Method to Produce HFC-143 with Improved Efficiency of Column 134

This example shows conversion of CFC-113 to HFC-143 in a first reactor and conversion of recycled reaction intermediates HCFC-123a, HCFC-133b, and HCFC-133 to HFC-143 in a second reactor. The overall layout and reaction system is same as Example 5, but with improved efficiency of distillation step performed in column 234 so that the stream 236 comprises HFC-143 with a purity greater than 95%, with less than 5% of impurities such as HCFC-133b, HCFC-133, HCC-160. The stream 238 (in the first run before recycling intermediates) includes the following mixture provided in Table 39 below.

TABLE 39
Composition of Bottom Stream 238 from Fourth Distillation Column 234

| mole % (desired) | | | | | | mole % (undesired) | | | | | | |
|---|---|---|---|---|---|---|---|---|---|---|---|---|
| R- | R- | R- | R- | R- | | R- | R- | R- | R- | R-142 | | |
| 143 | 133b | 133 | 123a | 113 | total | 143a | 170 | 152a | 160 | isomers | Other* | total |
| 31.46 | 48.83 | 6.25 | 9.93 | 0.00 | 96.47 | 0.00 | 0.00 | 0.00 | 0.00 | 3.00 | 0.53 | 3.53 |

The bottom stream 238 from the fourth distillation column 234 is also sent to the second reactor 240 of the two-reactor method. The second reactor 240 includes a one-inch 316 SS tube packed with 50 ml of 4% Pd/C catalyst. A thermocouple is inserted into the middle of the catalyst bed to read the operating temperature. The pressure control system consists of a RCV which controls the pressure by getting feedback from the pressure transducer placed after the second reactor 240. The second reactor 240 is operated at 45 psig, 280° C., with 10 g/h organic feed rate, and 150 ml/min H₂ feed rate.

The product composition in second product stream 242 from the second reactor 240 is determined using GC-FID analysis of the samples taken after the second reactor 240 using a sample bag filled with ˜50 g of water to capture hydrogen chloride (HCl) and hydrogen fluoride (HF). Before the GC analysis, the sample bag is heated at 60° C. for one hour to assure that all the organic content is in the gas phase.

Under these conditions, the conversion of HCFC-133b, HCFC-133, and HCFC-123a are 52%, 72%, and 100%, respectively, based on total moles of organic components of the composition. The chloroethane (HCC-160) and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) molecules are also converted to ethane (HC-170) and difluoroethane (HFC-152 isomers) in the reactor (˜90% conversion based on total moles of organic components of the composition).

The product stream 242 from the second reactor 240 (in the first run of recycling intermediates) includes the following mixture provided in Table 40 below.

TABLE 40
Composition of product stream from second reactor

| mole % (desired) | | | | | | mole % (undesired) | | | | | | |
|---|---|---|---|---|---|---|---|---|---|---|---|---|
| R- | R- | R- | R- | R- | | R- | R- | R- | R- | R-142 | | |
| 143 | 133b | 133 | 123a | 113 | total | 143a | 170 | 152a | 160 | isomers | Other* | total |
| 66.02 | 23.44 | 1.75 | 0.00 | 0.00 | 91.21 | 0.23 | 1.77 | 4.32 | 0.09 | 0.87 | 1.51 | 8.79 |

This product stream 242 from the second reactor 240 is mixed with the product stream 208 from the first reactor 206 and sent to the same distillation columns and scrubber as described above. The percent increase in R-143 is 110 mol %, relative to an amount of HFC-143 in the bottom stream 238 from the fourth distillation column 234 before entering the second reactor 240, as calculated from data in Table 39 and Table 40.

### Example 9

### Formation of HFO-1132E

The desired product (HFC-143) produced in Examples 1-5 is further reacted with a catalyst to produce HFO-1132E using Steps (ii) and (iii), as described above.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

### Aspects

Aspect 1 is a method for producing 1,1,2-trifluoroethane (HFC-143), comprising: reacting 1,1,2-trichloro-1,2,2-trifluoroethane (CFC-113) and at least one of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a) in the presence of a catalyst at a temperature of from 150° C. to 400° C. to produce 1,1,2-trifluoroethane (HFC-143).

Aspect 2 is the method of Aspect 1, wherein the catalyst is palladium metal supported on a carbon support.

Aspect 3 is the method of Aspect 2, wherein the catalyst comprises from about 1 wt. % to about 5 wt. % palladium metal supported on a carbon support.

Aspect 4 is the method of Aspect 2, wherein the catalyst comprises about 4 wt. % palladium metal supported on a carbon support.

Aspect 5 is the method of any one of Aspects 1-4, wherein the reacting step is conducted at a temperature of from 200° C. to 400° C., or from 225° C. to 350° C.

Aspect 6 is the method of any one of Aspect 1-5, wherein the reacting step is conducted at a temperature of from 250° C. to 325° C.

Aspect 7 is the method of any one of Aspects 1-6, wherein the reacting step produces a composition comprising: 45 mol % to 99.97 mol % 1,1,2-trifluoroethane (HFC-143); 0.01 mol % to 40 mol % 1-chloro-1,1,2-trifluoroethane (HCFC-133b); 0.01 mol % to 5 mol % 1-chloro-1,2,2-trifluoroethane (HCFC-133); and 0.01 mol % to 10 mol % 1,2-dichloro-1,1,2-trifluoroethane (HCFC-123a), based on the combined total moles of the HFC-143, HCFC-133b, HCFC-133, and HCFC-123a in the product composition.

Aspect 8 is the method of any one of Aspects 1-6, wherein the reacting step produces a composition comprising a total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) of at least 80 mol %, based on total moles of organic components of the composition.

Aspect 9 is the method of any one of Aspects 1-8, further comprising, after the reacting step, the additional step of recycling at least one of 1,1,2-trichloro-1,2,2-trifluoroethane (CFC-113), 1-chloro-1,1,2-trifluoroethane (HCFC-133b), 1-chloro-1,2,2-trifluoroethane (HCFC-133) and 1,2-dichloro-1,1,2-trifluoroethane (HCFC-123a) in the form of a recycling stream, back to the reacting step.

Aspect 10 is the method of Aspect 9, wherein the recycling stream comprises: 0.01 mol % to 20 mol % 1,1,2-trifluoroethane (HFC-143); 40 mol % to 99.93 mol % 1-chloro-1,1,2-trifluoroethane (HCFC-133b); 0.01 mol % to 20 mol % 1-chloro-1,2,2-trifluoroethane (HCFC-133); and 0.01 mol % to 20 mol % 1,2-dichloro-1,1,2-trifluoroethane (HCFC-123a), based on the combined total moles of the HFC-143, HCFC-133b, HCFC-133, and HCFC-123a in the recycling stream.

Aspect 11 is the method of any one of Aspects 1-10, further comprising reacting 1,1,2-trifluoroethane (HFC-143) with a catalyst to produce trans-1,2-difluoroethylene (HFO-1132E).

Aspect 12 is a method for producing 1,1,2-trifluoroethane (HFC-143), comprising reacting 1,1,2-trichloro-1,2,2-trifluoroethane (CFC-113) with hydrogen in the presence of a catalyst to produce a first product composition comprising 1,1,2-trifluoroethane (HFC-143) and at least one of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a); and reacting at least one of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a) from the first product composition with hydrogen in the presence of a catalyst to produce a second product composition comprising 1,1,2-trifluoroethane (HFC-143). The first reacting step may be conducted at a temperature of from 150° C. to 400° C., or from 200° C. to 400° C. The second reacting step may be conducted at a temperature of from 200° C. to 450° C., or from 225° C. to 400° C.

Aspect 13 is the method of Aspect 12, further comprising the additional steps, between the first reacting step and the second reacting step, of: removing hydrogen and acids to produce an essentially acid-free stream; and distilling the said essentially acid-free stream to produce an overhead composition comprising 1,1,2-trifluoroethane (HFC-143) and a bottom composition comprising 1,-chloro-1,1,2-trifluoroethane (CFC-133b).

Aspect 14 the method of Aspect 12 or Aspect 13, wherein the catalyst is palladium metal supported on a carbon support.

Aspect 15 is the method of Aspect 14, wherein the catalyst comprises about 4 wt. % palladium metal supported on a carbon support.

Aspect 16 is the method of any one of Aspects 12-15, wherein the second reacting step is conducted at a temperature of from 250° C. to 450° C.

Aspect 17 is the method of any one of Aspects 12-15, wherein the second reacting step is conducted at a temperature of from 275° C. to 400° C.

Aspect 18 is the method of any one of Aspects 12-17, wherein the second reacting step produces a composition comprising: 50 mol % to 99.97 mol % 1,1,2-trifluoroethane (HFC-143); 0.01 mol % to 40 mol % 1-chloro-1,1,2-trifluoroethane (HCFC-133b); 0.01 mol % to 10 mol % 1-chloro-1,2,2-trifluoroethane (HCFC-133); and 0.01 mol % to 10 mol % 1,2-dichloro-1,1,2-trifluoroethane (HCFC-123a), based on the combined total moles of the HFC-143, HCFC-133b, HCFC-133, and HCFC-123a in the composition.

Aspect 19 is the method of any one of Aspects 12-17, wherein the second reacting step produces a composition comprising a total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) of 80 mol % to 99.9 mol %, based on total moles of organic components of the composition.

Aspect 20 is the method of any one of Aspects 12-19, further comprising, after the second reacting step, the additional step of recycling at least one of 1,1,2-trichloro-1,2,2-trifluoroethane (CFC-113), 1-chloro-1,1,2-trifluoroethane (HCFC-133b), 1-chloro-1,2,2-trifluoroethane (HCFC-133) and 1,2-dichloro-1,1,2-trifluoroethane (HCFC-123a), in the form of a recycle stream, back to the reacting step.

Aspect 21 is the method of Aspect 20, wherein the recycle stream comprises: 50 mol % to 99.97 mol % 1,1,2-trifluoroethane (HFC-143); 0.01 mol % to 40 mol % 1-chloro-1,1,2-trifluoroethane (HCFC-133b); 0.01 mol % to 5 mol % 1-chloro-1,2,2-trifluoroethane (HCFC-133); and 0.01 mol % to 5 mol % 1,2-dichloro-1,1,2-trifluoroethane (HCFC-123a), based on the combined total moles of the HFC-143, HCFC-133b, HCFC-133, and HCFC-123a in the recycle stream.

Aspect 22 is the method of any one of Aspects 12-21, further comprising reacting at least one of the 1,1,2-trifluoroethane (HFC-143) of the first product composition and the 1,1,2-trifluoroethane (HFC-143) of the second product composition with a catalyst to produce trans-1,2-difluoroethylene (HFO-1132E).

Aspect 23 is the method of any one of Aspects 1-8, wherein the catalyst comprises from about 0.5 wt. % to about 10 wt. % palladium metal supported on a carbon support; wherein the reacting step is conducted at a temperature of from about 200° C. to about 350° C.

Aspect 24 is the method of any one of Aspects 1-8, wherein the catalyst comprises from about 0.5 wt. % to about 10 wt. % palladium metal supported on a carbon support; wherein the reacting step is conducted at a temperature of from about 230° C. to about 290° C.

Aspect 25 is the method of any one of Aspects 1-8, wherein the catalyst comprises from about 0.5 wt. % to about 10 wt. % palladium metal supported on a carbon support; wherein the reacting step is conducted at a temperature of from about 250° C. to about 280° C.

Aspect 26 is the method of any one of Aspects 1-8, wherein the catalyst comprises from about 0.5 wt. % to about 10 wt. % palladium metal supported on a carbon support; wherein the reacting step is conducted at a temperature of from about 250° C. to about 350° C.

Aspect 27 is the method of any one of Aspects 1-8, wherein the catalyst comprises from about 1 wt. % to about 5 wt. % palladium metal supported on a carbon support; wherein the reacting step is conducted at a temperature of from about 200° C. to about 350° C.

Aspect 28 is the method of any one of Aspects 1-8, wherein the catalyst comprises from about 1 wt. % to about 5 wt. % palladium metal supported on a carbon support; wherein the reacting step is conducted at a temperature of from about 230° C. to about 290° C.

Aspect 29 is the method of any one of Aspects 1-8, wherein the catalyst comprises from about 1 wt. % to about 5 wt. % palladium metal supported on a carbon support; wherein the reacting step is conducted at a temperature of from about 250° C. to about 280° C.

Aspect 30 is the method of any one of Aspects 1-8, wherein the catalyst comprises from about 1 wt. % to about 5 wt. % palladium metal supported on a carbon support; wherein the reacting step is conducted at a temperature of from about 250° C. to about 350° C.

Aspect 31 is the method of any one of Aspects 1-8, wherein the catalyst comprises from about 0.1 wt. % to about 10 wt. % palladium metal supported on an alpha alumina support; wherein the reacting step is conducted at a temperature of from about 200° C. to about 350° C.

Aspect 32 is the method of any one of Aspects 1-8, wherein the catalyst comprises from about 0.1 wt. % to about 10 wt. % palladium metal supported on an alpha alumina support; wherein the reacting step is conducted at a temperature of from about 230° C. to about 290° C.

Aspect 33 is the method of any one of Aspects 1-8, wherein the catalyst comprises from about 0.1 wt. % to about 10 wt. % palladium metal supported on an alpha alumina support; wherein the reacting step is conducted at a temperature of from about 250° C. to about 280° C.

Aspect 34 is the method of any one of Aspects 1-8, wherein the catalyst comprises from about 0.1 wt. % to about 10 wt. % palladium metal supported on an alpha alumina support; wherein the reacting step is conducted at a temperature of from about 250° C. to about 350° C.

Aspect 35 is the method of any one of Aspects 1-8, wherein the catalyst comprises from about 0.2 wt. % to about 5 wt. % palladium metal supported on an alpha alumina support; wherein the reacting step is conducted at a temperature of from about 200° C. to about 350° C.

Aspect 36 is the method of any one of Aspects 1-8, wherein the catalyst comprises from about 0.2 wt. % to about 5 wt. % palladium metal supported on an alpha alumina support; wherein the reacting step is conducted at a temperature of from about 230° C. to about 290° C.

Aspect 37 is the method of any one of Aspects 1-8, wherein the catalyst comprises from about 0.2 wt. % to about 5 wt. % palladium metal supported on an alpha alumina support; wherein the reacting step is conducted at a temperature of from about 250° C. to about 280° C.

Aspect 38 is the method of any one of Aspects 1-8, wherein the catalyst comprises from about 0.2 wt. % to about 5 wt. % palladium metal supported on an alpha alumina support; wherein the reacting step is conducted at a temperature of from about 250° C. to about 350° C.

Aspect 39 is the method of any one of Aspects 12-19, wherein for the first reacting step, the catalyst comprises from about 0.5 wt. % to about 10 wt. % palladium metal supported on a carbon support; wherein the first reacting step is conducted at a temperature of from about 200° C. to about 400° C.

Aspect 40 is the method of any one of Aspects 12-19, wherein for the first reacting step, the catalyst comprises from about 0.5 wt. % to about 10 wt. % palladium metal supported on a carbon support; wherein the first reacting step is conducted at a temperature of from about 220° C. to about 400° C.

Aspect 41 is the method of any one of Aspects 12-19, wherein for the first reacting step, the catalyst comprises from about 0.5 wt. % to about 10 wt. % palladium metal supported on a carbon support; wherein the first reacting step is conducted at a temperature of from about 240° C. to about 350° C.

Aspect 42 is the method of any one of Aspects 12-19, wherein for the first reacting step, the catalyst comprises from about 0.5 wt. % to about 10 wt. % palladium metal supported on a carbon support; wherein the first reacting step is conducted at a temperature of from about 250° C. to about 300° C.

Aspect 43 is the method of any one of Aspects 12-19, wherein for the first reacting step, the catalyst comprises from about 1 wt. % to about 5 wt. % palladium metal supported on a carbon support; wherein the first reacting step is conducted at a temperature of from about 200° C. to about 400° C.

Aspect 44 is the method of any one of Aspects 12-19, wherein for the first reacting step, the catalyst comprises from about 1 wt. % to about 5 wt. % palladium metal supported on a carbon support; wherein the first reacting step is conducted at a temperature of from about 220° C. to about 400° C.

Aspect 45 is the method of any one of Aspects 12-19, wherein for the first reacting step, the catalyst comprises from about 1 wt. % to about 5 wt. % palladium metal supported on a carbon support; wherein the first reacting step is conducted at a temperature of from about 240° C. to about 350° C.

Aspect 46 is the method of any one of Aspects 12-19, wherein for the first reacting step, the catalyst comprises from about 1 wt. % to about 5 wt. % palladium metal supported on a carbon support; wherein the first reacting step is conducted at a temperature of from about 250° C. to about 300° C.

Aspect 47 is the method of any one of Aspects 12-19, wherein for the first reacting step, the catalyst comprises from about 2 wt. % to about 4 wt. % palladium metal supported on a carbon support; wherein the first reacting step is conducted at a temperature of from about 200° C. to about 400° C.

Aspect 48 is the method of any one of Aspects 12-19, wherein for the first reacting step, the catalyst comprises from about 2 wt. % to about 4 wt. % palladium metal supported on a carbon support; wherein the first reacting step is conducted at a temperature of from about 220° C. to about 400° C.

Aspect 49 is the method of any one of Aspects 12-19, wherein for the first reacting step, the catalyst comprises from about 2 wt. % to about 4 wt. % palladium metal supported on a carbon support; wherein the first reacting step is conducted at a temperature of from about 240° C. to about 350° C.

Aspect 50 is the method of any one of Aspects 12-19, wherein for the first reacting step, the catalyst comprises from about 2 wt. % to about 4 wt. % palladium metal supported on a carbon support; wherein the first reacting step is conducted at a temperature of from about 250° C. to about 300° C.

Aspect 51 is the method of any one of Aspects 12-19, wherein for the first reacting step, the catalyst comprises from about 0.1 wt. % to about 10 wt. % palladium metal supported on an alpha alumina support; wherein the first reacting step is conducted at a temperature of from about 150° C. to about 350° C.

Aspect 52 is the method of any one of Aspects 12-19, wherein for the first reacting step, the catalyst comprises from about 0.1 wt. % to about 10 wt. % palladium metal supported on an alpha alumina support; wherein the first reacting step is conducted at a temperature of from about 150° C. to about 250° C.

Aspect 53 is the method of any one of Aspects 12-19, wherein for the first reacting step, the catalyst comprises from about 0.2 wt. % to about 5 wt. % palladium metal supported on an alpha alumina support; wherein the first reacting step is conducted at a temperature of from about 150° C. to about 350° C.

Aspect 54 is the method of any one of Aspects 12-19, wherein for the first reacting step, the catalyst comprises from about 0.2 wt. % to about 5 wt. % palladium metal supported on an alpha alumina support; wherein the first reacting step is conducted at a temperature of from about 150° C. to about 250° C.

Aspect 55 is the method of any one of Aspects 12-19, wherein for the second reacting step, the catalyst comprises from about 0.1 wt. % to about 10 wt. % palladium metal supported on a carbon support; wherein the second reacting step is conducted at a temperature of from about 200° C. to about 450° C.

Aspect 56 is the method of any one of Aspects 12-19, wherein for the second reacting step, the catalyst comprises from about 0.1 wt. % to about 10 wt. % palladium metal supported on a carbon support; wherein the second reacting step is conducted at a temperature of from about 220° C. to about 400° C.

Aspect 57 is the method of any one of Aspects 12-19, wherein for the second reacting step, the catalyst comprises from about 0.1 wt. % to about 10 wt. % palladium metal supported on a carbon support; wherein the second reacting step is conducted at a temperature of from about 250° C. to about 350° C.

Aspect 58 is the method of any one of Aspects 12-19, wherein for the second reacting step, the catalyst comprises from about 0.1 wt. % to about 10 wt. % palladium metal supported on a carbon support; wherein the second reacting step is conducted at a temperature of from about 275° C. to about 350° C.

Aspect 59 is the method of any one of Aspects 12-19, wherein for the second reacting step, the catalyst comprises from about 0.5 wt. % to about 5 wt. % palladium metal supported on a carbon support; wherein the second reacting step is conducted at a temperature of from about 200° C. to about 450° C.

Aspect 60 is the method of any one of Aspects 12-19, wherein for the second reacting step, the catalyst comprises from about 0.5 wt. % to about 5 wt. % palladium metal supported on a carbon support; wherein the second reacting step is conducted at a temperature of from about 220° C. to about 400° C.

Aspect 61 is the method of any one of Aspects 12-19, wherein for the second reacting step, the catalyst comprises from about 0.5 wt. % to about 5 wt. % palladium metal supported on a carbon support; wherein the second reacting step is conducted at a temperature of from about 250° C. to about 350° C.

Aspect 62 is the method of any one of Aspects 12-19, wherein for the second reacting step, the catalyst comprises from about 0.5 wt. % to about 5 wt. % palladium metal supported on a carbon support; wherein the second reacting step is conducted at a temperature of from about 275° C. to about 350° C.

Aspect 63 is the method of any one of Aspects 12-19, wherein for the second reacting step, the catalyst comprises from about 1 wt. % to about 4 wt. % palladium metal supported on a carbon support; wherein the second reacting step is conducted at a temperature of from about 200° C. to about 450° C.

Aspect 64 is the method of any one of Aspects 12-19, wherein for the second reacting step, the catalyst comprises from about 1 wt. % to about 4 wt. % palladium metal supported on a carbon support; wherein the second reacting step is conducted at a temperature of from about 220° C. to about 400° C.

Aspect 65 is the method of any one of Aspects 12-19, wherein for the second reacting step, the catalyst comprises from about 1 wt. % to about 4 wt. % palladium metal supported on a carbon support; wherein the second reacting step is conducted at a temperature of from about 250° C. to about 350° C.

Aspect 66 is the method of any one of Aspects 12-19, wherein for the second reacting step, the catalyst comprises from about 1 wt. % to about 4 wt. % palladium metal supported on a carbon support; wherein the second reacting step is conducted at a temperature of from about 275° C. to about 350° C.

Aspect 67 is the method of any one of Aspects 12-19, wherein for the second reacting step, the catalyst comprises from about 0.1 wt. % to about 10 wt. % palladium metal supported on an alpha alumina support; wherein the second reacting step is conducted at a temperature of from about 150° C. to about 350° C.

Aspect 68 is the method of any one of Aspects 12-19, wherein for the second reacting step, the catalyst comprises from about 0.1 wt. % to about 10 wt. % palladium metal supported on an alpha alumina support; wherein the second reacting step is conducted at a temperature of from about 150° C. to about 250° C.

Aspect 69 is the method of any one of Aspects 12-19, wherein for the second reacting step, the catalyst comprises from about 0.2 wt. % to about 5 wt. % palladium metal supported on an alpha alumina support; wherein the second reacting step is conducted at a temperature of from about 150° C. to about 350° C.

Aspect 71 is the method of any one of Aspects 12-19, wherein for the second reacting step, the catalyst comprises from about 0.2 wt. % to about 5 wt. % palladium metal supported on an alpha alumina support; wherein the second reacting step is conducted at a temperature of from about 150° C. to about 250° C.

Aspect 72 is a composition comprising: 45 mol % to 99.97 mol % 1,1,2-trifluoroethane (HFC-143); 0.01 mol % to 40 mol % 1-chloro-1,1,2-trifluoroethane (HCFC-133b); 0.01 mol % to 5 mol % 1-chloro-1,2,2-trifluoroethane (HCFC-133); and 0.01 mol % to 10 mol % 1,2-dichloro-1,1,2-trifluoroethane (HCFC-123a), based on the combined total moles of the HFC-143, HCFC-133b, HCFC-133, and HCFC-123a in the composition.

Aspect 73 is a composition comprising: 0.01 mol % to 20 mol % 1,1,2-trifluoroethane (HFC-143); 40 mol % to 99.97 mol % 1-chloro-1,1,2-trifluoroethane (HCFC-133b); 0.01 mol % to 20 mol % 1-chloro-1,2,2-trifluoroethane (HCFC-133); and 0.01 mol % to 20 mol % 1,2-dichloro-1,1,2-trifluoroethane (HCFC-123a), based on the combined total moles of the HFC-143, HCFC-133b, HCFC-133, and HCFC-123a in the composition.

Aspect 74 is a composition comprising: 0.01 mol % to 50 mol % 1,1,2-trifluoroethane (HFC-143); 20 mol % to 99.97 mol % 1-chloro-1,1,2-trifluoroethane (HCFC-133b); 0.01 mol % to 10 mol % 1-chloro-1,2,2-trifluoroethane (HCFC-133); and 0.01 mol % to 20 mol % 1,2-dichloro-1,1,2-trifluoroethane (HCFC-123a), based on the combined total moles of the HFC-143, HCFC-133b, HCFC-133, and HCFC-123a in the composition.

Aspect 75 is a composition comprising: 40 mol % to 99.97 mol % 1,1,2-trifluoroethane (HFC-143); 0.01 mol % to 40 mol % 1-chloro-1,1,2-trifluoroethane (HCFC-133b); 0.01 mol % to 10 mol % 1-chloro-1,2,2-trifluoroethane (HCFC-133); and 0.01 mol % to 10 mol % 1,2-dichloro-1,1,2-trifluoroethane (HCFC-123a), based on the combined total moles of the HFC-143, HCFC-133b, HCFC-133, and HCFC-123a in the composition.

Aspect 76 is a composition comprising: 50 mol % to 99.97 mol % 1,1,2-trifluoroethane (HFC-143); 0.01 mol % to 40 mol % 1-chloro-1,1,2-trifluoroethane (HCFC-133b); 0.01 mol % to 5 mol % 1-chloro-1,2,2-trifluoroethane (HCFC-133); and 0.01 mol % to 5 mol % 1,2-dichloro-1,1,2-trifluoroethane (HCFC-123a), based on the combined total moles of the HFC-143, HCFC-133b, HCFC-133, and HCFC-123a in the composition.

Aspect 77 is the method of Aspect 12, wherein the first reacting step produces a composition comprising: 45 mol % to 99.97 mol % 1,1,2-trifluoroethane (HFC-143); 0.01 mol % to 40 mol % 1-chloro-1,1,2-trifluoroethane (HCFC-133b); 0.01 mol % to 5 mol % 1-chloro-1,2,2-trifluoroethane (HCFC-133); and 0.01 mol % to 10 mol % 1,2-dichloro-1,1,2-trifluoroethane (HCFC-123a), based on the combined total moles of the HFC-143, HCFC-133b, HCFC-133, and HCFC-123a in the composition.

Aspect 78 is the method of Aspect 13, wherein the distilling step produces a first distilled composition; wherein the second reacting step produces a second composition; wherein an amount of 1,1,2-trifluoroethane (HFC-143) in the second composition is increased by 35 mol % to 130 mol % relative to an amount of 1,1,2-trifluoroethane (HFC-143) in the first distilled composition.

Aspect 79 is the method of Aspect 13, wherein the distilling step produces a first distilled composition; wherein the second reacting step produces a second composition; wherein an amount of 1,1,2-trifluoroethane (HFC-143) in the second composition is increased by 40 mol % to 130 mol % relative to an amount of 1,1,2-trifluoroethane (HFC-143) in the first distilled composition.

Aspect 80 is the method of Aspect 13, wherein the distilling step produces a distilled composition comprising: 0.01 mol % to 50 mol % 1,1,2-trifluoroethane (HFC-143); 20 mol % to 99.97 mol % 1-chloro-1,1,2-trifluoroethane (HCFC-133b); 0.01 mol % to 10 mol % 1-chloro-1,2,2-trifluoroethane (HCFC-133); and 0.01 mol % to 20 mol % 1,2-dichloro-1,1,2-trifluoroethane (HCFC-123a), based on the combined total moles of the HFC-143, HCFC-133b, HCFC-133, and HCFC-123a in the distilled composition.

Aspect 81 is the method of any one of Aspects 1-8, wherein the catalyst comprises from about 0.5 wt. % to about 10 wt. % rhodium metal supported on a carbon support; wherein the reacting step is conducted at a temperature of from about 200° C. to about 350° C.

Aspect 82 is the method of any one of Aspects 1-8, wherein the catalyst comprises from about 0.5 wt. % to about 10 wt. % rhodium metal supported on a carbon support; wherein the reacting step is conducted at a temperature of from about 230° C. to about 290° C.

Aspect 83 is the method of any one of Aspects 1-8, wherein the catalyst comprises from about 0.5 wt. % to about 10 wt. % rhodium metal supported on a carbon support; wherein the reacting step is conducted at a temperature of from about 250° C. to about 280° C.

Aspect 84 is the method of any one of Aspects 1-8, wherein the catalyst comprises from about 0.5 wt. % to about 10 wt. % rhodium metal supported on a carbon support; wherein the reacting step is conducted at a temperature of from about 250° C. to about 350° C.

Aspect 85 is the method of any one of Aspects 1-8, wherein the catalyst comprises from about 1 wt. % to about 5 wt. % rhodium metal supported on a carbon support; wherein the reacting step is conducted at a temperature of from about 200° C. to about 350° C.

Aspect 86 is the method of any one of Aspects 1-8, wherein the catalyst comprises from about 1 wt. % to about 5 wt. % rhodium metal supported on a carbon support; wherein the reacting step is conducted at a temperature of from about 230° C. to about 290° C.

Aspect 87 is the method of any one of Aspects 1-8, wherein the catalyst comprises from about 1 wt. % to about 5 wt. % rhodium metal supported on a carbon support; wherein the reacting step is conducted at a temperature of from about 250° C. to about 280° C.

Aspect 88 is the method of any one of Aspects 1-8, wherein the catalyst comprises from about 1 wt. % to about 5 wt. % rhodium metal supported on a carbon support; wherein the reacting step is conducted at a temperature of from about 250° C. to about 350° C.

Aspect 89 is the method of any one of Aspects 1-8, wherein the catalyst comprises from about 0.1 wt. % to about 10 wt. % rhodium metal supported on an alpha alumina support; wherein the reacting step is conducted at a temperature of from about 200° C. to about 350° C.

Aspect 90 is the method of any one of Aspects 1-8, wherein the catalyst comprises from about 0.1 wt. % to about 10 wt. % rhodium metal supported on an alpha alumina support; wherein the reacting step is conducted at a temperature of from about 230° C. to about 290° C.

Aspect 91 is the method of any one of Aspects 1-8, wherein the catalyst comprises from about 0.1 wt. % to about 10 wt. % rhodium metal supported on an alpha alumina support; wherein the reacting step is conducted at a temperature of from about 250° C. to about 280° C.

Aspect 92 is the method of any one of Aspects 1-8, wherein the catalyst comprises from about 0.1 wt. % to about 10 wt. % rhodium metal supported on an alpha alumina support; wherein the reacting step is conducted at a temperature of from about 250° C. to about 350° C.

Aspect 93 is the method of any one of Aspects 1-8, wherein the catalyst comprises from about 0.2 wt. % to about 5 wt. % rhodium metal supported on an alpha alumina support; wherein the reacting step is conducted at a temperature of from about 200° C. to about 350° C.

Aspect 94 is the method of any one of Aspects 1-8, wherein the catalyst comprises from about 0.2 wt. % to about 5 wt. % rhodium metal supported on an alpha alumina support; wherein the reacting step is conducted at a temperature of from about 230° C. to about 290° C.

Aspect 95 is the method of any one of Aspects 1-8, wherein the catalyst comprises from about 0.2 wt. % to about 5 wt. % rhodium metal supported on an alpha alumina support; wherein the reacting step is conducted at a temperature of from about 250° C. to about 280° C.

Aspect 96 is the method of any one of Aspects 1-8, wherein the catalyst comprises from about 0.2 wt. % to about 5 wt. % rhodium metal supported on an alpha alumina support; wherein the reacting step is conducted at a temperature of from about 250° C. to about 350° C.

Aspect 97 is the method of any one of Aspects 12-19, wherein for the first reacting step, the catalyst comprises from about 0.5 wt. % to about 10 wt. % rhodium metal supported on a carbon support; wherein the first reacting step is conducted at a temperature of from about 200° C. to about 400° C.

Aspect 98 is the method of any one of Aspects 12-19, wherein for the first reacting step, the catalyst comprises from about 1 wt. % to about 5 wt. % rhodium metal supported on a carbon support; wherein the first reacting step is conducted at a temperature of from about 250° C. to about 350° C.

Aspect 99 is the method of any one of Aspects 12-19, wherein for the second reacting step, the catalyst comprises from about 0.1 wt. % to about 10 wt. % rhodium metal supported on a carbon support; wherein the second reacting step is conducted at a temperature of from about 200° C. to about 450° C.

Aspect 100 is the method of any one of Aspects 12-19, wherein for the second reacting step, the catalyst comprises from about 1 wt. % to about 4 wt. % rhodium metal supported on a carbon support; wherein the second reacting step is conducted at a temperature of from about 250° C. to about 350° C.

What is claimed is:

1. A method for producing 1,1,2-trifluoroethane (HFC-143), comprising:
reacting 1,1,2-trichloro-1,2,2-trifluoroethane (CFC-113) and at least one of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a) in the presence of a catalyst at a temperature of from 150° C. to 400° C. to produce 1,1,2-trifluoroethane (HFC-143).

2. The method of claim 1, wherein the catalyst is palladium metal supported on a carbon support.

3. The method of claim 2, wherein the catalyst comprises from about 1 wt. % to about 5 wt. % palladium metal supported on a carbon support.

4. The method of claim 1, wherein the reacting step is conducted at a temperature of from 200° C. to 400° C.

5. The method of claim 1, wherein the reacting step produces a composition comprising:
45 mol % to 99.97 mol % 1,1,2-trifluoroethane (HFC-143);
0.01 mol % to 40 mol % 1-chloro-1,1,2-trifluoroethane (HCFC-133b);
0.01 mol % to 5 mol % 1-chloro-1,2,2-trifluoroethane (HCFC-133); and
0.01 mol % to 10 mol % 1,2-dichloro-1,1,2-trifluoroethane (HCFC-123a), based on the combined total moles of the HFC-143, HCFC-133b, HCFC-133, and HCFC-123a in the product composition.

6. The method of claim 1, wherein the reacting step produces a composition comprising a total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) of at least 80 mol %, based on total moles of organic components of the composition.

7. The method of claim 1, further comprising, after the reacting step, the additional step of recycling at least one of 1,1,2-trichloro-1,2,2-trifluoroethane (CFC-113), 1-chloro-1,1,2-trifluoroethane (HCFC-133b), 1-chloro-1,2,2-trifluoroethane (HCFC-133) and 1,2-dichloro-1,1,2-trifluoroethane (HCFC-123a) in the form of a recycling stream, back to the reacting step.

8. The method of claim 7, wherein the recycling stream comprises:
0.01 mol % to 20 mol % 1,1,2-trifluoroethane (HFC-143);
40 mol % to 99.97 mol % 1-chloro-1,1,2-trifluoroethane (HCFC-133b);
0.01 mol % to 20 mol % 1-chloro-1,2,2-trifluoroethane (HCFC-133); and
0.01 mol % to 20 mol % 1,2-dichloro-1,1,2-trifluoroethane (HCFC-123a), based on the combined total moles of the HFC-143, HCFC-133b, HCFC-133, and HCFC-123a in the recycling stream.

9. The method of claim 1, further comprising reacting 1,1,2-trifluoroethane (HFC-143) with a catalyst to produce trans-1,2-difluoroethylene (HFO-1132E).

10. A method for producing 1,1,2-trifluoroethane (HFC-143), comprising:
reacting 1,1,2-trichloro-1,2,2-trifluoroethane (CFC-113) with hydrogen in the presence of a catalyst to produce a first product composition comprising 1,1,2-trifluoroethane (HFC-143) and at least one of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a); and
reacting at least one of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a) from the first product composition with hydrogen in the presence of a catalyst to produce a second product composition comprising 1,1,2-trifluoroethane (HFC-143).

11. The method of claim 10, wherein the first reacting step is conducted at a temperature of from 150° C. to 400° C.;
wherein the second reacting step is conducted at a temperature of from 200° C. to 450° C.

12. The method of claim 10, wherein the first reacting step produces a composition comprising:
45 mol % to 99.97 mol % 1,1,2-trifluoroethane (HFC-143);
0.01 mol % to 40 mol % 1-chloro-1,1,2-trifluoroethane (HCFC-133b);
0.01 mol % to 5 mol % 1-chloro-1,2,2-trifluoroethane (HCFC-133); and
0.01 mol % to 10 mol % 1,2-dichloro-1,1,2-trifluoroethane (HCFC-123a), based on the combined total moles of the HFC-143, HCFC-133b, HCFC-133, and HCFC-123a in the composition.

13. The method of claim 10, further comprising, between the first reacting step and the second reacting step:
removing hydrogen and acid to produce an essentially acid-free stream; and
distilling the essentially acid-free stream to produce a first distilled composition comprising 1,1,2-trifluoroethane (HFC-143);
wherein an amount of 1,1,2-trifluoroethane (HFC-143) in the second product composition is increased by from about 35 mol % to about 130 mol % relative to an amount of 1,1,2-trifluoroethane (HFC-143) in the first distilled composition.

14. The method of claim 13, wherein the distilling step produces a distilled composition comprising:
0.01 mol % to 50 mol % 1,1,2-trifluoroethane (HFC-143);
20 mol % to 99.97 mol % 1-chloro-1,1,2-trifluoroethane (HCFC-133b);
0.01 mol % to 10 mol % 1-chloro-1,2,2-trifluoroethane (HCFC-133); and
0.01 mol % to 20 mol % 1,2-dichloro-1,1,2-trifluoroethane (HCFC-123a), based on the combined total moles of the HFC-143, HCFC-133b, HCFC-133, and HCFC-123a in the composition.

15. The method of claim 10, wherein the catalyst is palladium metal supported on a carbon support.

16. The method of claim 10, wherein the second reacting step produces a composition comprising:
50 mol % to 99.97 mol % 1,1,2-trifluoroethane (HFC-143);
0.01 mol % to 40 mol % 1-chloro-1,1,2-trifluoroethane (HCFC-133b);
0.01 mol % to 5 mol % 1-chloro-1,2,2-trifluoroethane (HCFC-133); and
0.01 mol % to 5 mol % 1,2-dichloro-1,1,2-trifluoroethane (HCFC-123a), based on the combined total moles of the HFC-143, HCFC-133b, HCFC-133, and HCFC-123a in the composition.

17. The method of claim 10, wherein the second reacting step produces a composition comprising a total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) of at least 80 mol %, based on total moles of organic components of the composition.

18. The method of claim 10, further comprising, after the second reacting step, the additional step of recycling at least one of 1,1,2-trichloro-1,2,2-trifluoroethane (CFC-113), 1-chloro-1,1,2-trifluoroethane (HCFC-133b), 1-chloro-1,2,2-trifluoroethane (HCFC-133) and 1,2-dichloro-1,1,2-trifluoroethane (HCFC-123a), in the form of a recycle stream, back to the reacting step.

19. The method of claim 18, wherein the recycle stream comprises:
50 mol % to 99.97 mol % 1,1,2-trifluoroethane (HFC-143);
0.01 mol % to 40 mol % 1-chloro-1,1,2-trifluoroethane (HCFC-133b);
0.01 mol % to 5 mol % 1-chloro-1,2,2-trifluoroethane (HCFC-133); and
0.01 mol % to 5 mol % 1,2-dichloro-1,1,2-trifluoroethane (HCFC-123a), based on the combined total moles of the HFC-143, HCFC-133b, HCFC-133, and HCFC-123a in the recycle stream.

20. The method of claim 10, further comprising reacting at least one of the 1,1,2-trifluoroethane (HFC-143) of the first product composition and the 1,1,2-trifluoroethane (HFC-143) of the second product composition with a catalyst to produce trans-1,2-difluoroethylene (HFO-1132E).
